SEQUENCE LISTING (8 sequences)

SEQ ID NO: 1 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 75 aa, Severe acute respiratory syndrome-related coronavirus
MYSFVSEETGTLIVNSVLLFLAFVVFLLVTLAILTALRLCAYCCNIVNVSLVKPSFYVYSRVKNLNSSRVPDLLV

SEQ ID NO: 3 - protein, 222 aa, Severe acute respiratory syndrome-related coronavirus
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 4 - protein, 419 aa, Severe acute respiratory syndrome-related coronavirus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 5 - DNA, 29903 bp, Severe acute respiratory syndrome-related coronavirus
  source 1..29903: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 6 - DNA, 18 bp, synthetic construct
  source 1..18: mol_type = other DNA
agctggcgcagattccag

SEQ ID NO: 7 - DNA, 21 bp, synthetic construct
  source 1..21: mol_type = other DNA
cctccaatttccgcgtgcagc

SEQ ID NO: 8 - DNA, 24 bp, synthetic construct
  source 1..24: mol_type = other DNA
gttccggtcaggccgttgaaattg

(19) United States
(12) Patent Application Publication
(10) Pub. No.: US 2024/0318266 A1
(43) Pub. Date: Sep. 26, 2024

(54) HIGH-THROUGHPUT SCREENING OF SARS-COV-2 VARIANTS

(71) Applicant: The J. David Gladsone Institutes, a Testamentary Trust Esablished under the will of J. David Gladsto, San Francisco, CA (US)

(72) Inventors: Taha Yasin Taha, Walnut Creek, CA (US); Melanie Ott, Mill Valley, CA (US)

(21) Appl. No.: 18/615,828

(22) Filed: Mar. 25, 2024

Related U.S. Application Data
(60) Provisional application No. 63/454,355, filed on Mar. 24, 2023.

(51) Int. Cl. C12Q 1/70; C07K 14/005; C12N 7/00; C12N 15/10

(52) U.S. Cl. CPC C12Q 1/70; C07K 14/005; C12N 7/00; C12N 15/1058; C12N 15/1096; C12N 2770/20022; C12N 2770/20023

(57) ABSTRACT

Provided herein are compositions and methods to identify mutations in one or more SARS-CoV-2 structural proteins that affect infectivity.

### PRIORITY

This application claims the benefit of U.S. Provisional Application No. 63/454,355, filed Mar. 24, 2023, the content of which is herein incorporated by reference in its entirety.

### INCORPORATION BY REFERENCE OF SEQUENCE LISTING

This application contains a Sequence Listing which has been submitted electronically in ST26 format and hereby incorporated by reference in its entirety. Said ST26 file, created on May 30, 2024, is name 3730217US1.xml and is 47,285 bytes in size.

### BACKGROUND

The COVID-19 pandemic is a leading cause of death globally, owing to the ongoing emergence of severe acute respiratory syndrome coronavirus 2 (SARS-CoV-2) variants with increased transmissibility and antibody neutralization escape. Understanding the molecular determinants of enhanced infectivity is central to vaccine and therapeutic development, but research is hindered because SARS-CoV-2 can be studied only in a biosafety level 3 (BSL-3) laboratory. Furthermore, technical challenges impede efforts to generate mutant infectious clones of SARS-CoV-2 (1-5). Current studies employ Spike (S) protein pseudotyped lentivirus systems for evaluation of S-mediated ACE2 receptor binding and cell entry (6,7). However, many mutations in circulating variants occur outside of the S gene and are thus inaccessible by this approach (8).

### SUMMARY

Given the large sequence space accessible to viruses for mutations, it is nearly impossible to use a brute force approach to test variations at amino acid positions in a rapid fashion that can be useful to recommend vaccine approaches. Thus, there is a need for improved, preemptive variant prediction or assessment prior to widespread infections in the population. Provided herein are methods and compositions to overcome current technical challenges by using a sequencing-based high-throughput approach to screen hundreds of thousands to millions of SARS-CoV-2 variants in a fast 2-3-month turnaround time. The iterative nature of the approach allows for continued screening and identification of future variants of concern prior to widespread infections in the population.

One embodiment provides a high throughput method to identify mutations in one or more SARS-CoV-2 structural proteins that affect infectivity comprising: a) obtaining a library of mutants for at least one SARS-CoV-2 structural protein, b) generating a virus like particle (VLP) pool by transducing plasmids expressing SARS-CoV-2 structural proteins E, M, N and S and a cis-acting RNA sequence that triggers packaging in initial cells, wherein at least one structural protein in each VLP has at least one of the mutants from the library of a), c) infecting secondary cells by contacting said secondary cells with supernatant from the initial cell culture of b), and d) sequencing, such as PacBio or other massively parallel sequencing, the viral sequences in the initial and secondary cells to identify mutations in the said at least one structural protein that affect the infectivity, wherein the viral sequences in the secondary cells of c) correlate with infectivity and the viral sequences in the initial cells of b) but not c) correlate with decreased infectivity.

Another embodiment provides a high throughout method to identify mutations in one or more SARS-CoV-2 structural proteins that affect sensitivity of said virus to a selection pressure comprising: a) obtaining a library of mutants for at least one SARS-CoV-2 structural protein, b) generating a virus like particle (VLP) pool by transducing plasmids expressing SARS-CoV-2 structural proteins E, M, N and S and a cis-acting RNA sequence that triggers packaging into initial cells and culturing, wherein at least one structural protein in each VLP has at least one of the mutants from the library of a), c) infecting secondary cells by contacting said secondary cells with supernatant from the initial cell culture of b), wherein the initial cells are exposed to a selection pressure prior to infecting said secondary cells, and d) sequencing, such as by PacBio, or other massively parallel sequencing method, the viral sequences in the secondary cells to identify mutations in the said at least one structural protein that affect the sensitivity of said virus to said selection pressure.

Another embodiment provides a high throughput method to map escape sites in an epitope of a structural SARS-CoV-2 protein from neutralizing antibodies comprising: a) obtaining a library of mutants for at least one SARS-CoV-2 structural protein, b) generating a virus like particles (VLP) pool by transducing plasmids expressing SARS-CoV-2 structural proteins E, M, N and S and a cis-acting RNA sequence that triggers packaging into initial cells and culturing, wherein at least one structural protein in each VLP has at least one of the mutants from the library of a), c) infecting secondary cells by contacting said secondary cells with supernatant from the initial cell culture of b), wherein the initial cells are exposed to neutralizing antibodies prior to infecting said secondary cells, and d) sequencing, such as by PacBio sequencing, or other massively parallel sequence method, the viral sequences in the secondary cells to identify mutations in the said at least one structural protein that escaped the neutralizing antibodies.

In some embodiments, the mutant structural protein is S, N, M, E protein or a combination thereof. In some embodiments, the cis-acting RNA sequence is PS9 or T20. In some embodiment, the initial and secondary cells are from human, bat, bird, or dog. In some embodiments, the initial and secondary cells are from a cell line. In other embodiments, the initial and secondary cells are kidney cells. In some embodiments, the secondary cells overexpress the ACE2 and/or TMPRSS2. In some embodiments, the initial and secondary cells are human.

In some embodiments, the selection pressure is a therapeutic compound. In some embodiments, the therapeutic compound is an antibody or sera from a human following infection or vaccination. In some embodiments, the therapeutic compound is a small molecule, a protein, a peptide, a polynucleotide, a polysaccharide, an oil, a solution or a plant extract. In some embodiments, the small molecule is an antiviral compound. In some embodiments, the neutralizing antibodies are from sera from a human following infection or vaccination.

In some embodiments, prior to sequencing the viral sequences in d), the RNA is extracted, RT-PCT is performed on said RNA, and sequencing adapters are added to the DNA obtained from the RT-PCR.

### BRIEF DESCRIPTION OF THE DRAWINGS

FIGS. 1A-1F demonstrate the development of a virus-like particle system to package Spike transcripts. (A) Schematic of validation experiment. (B) Spike RBD RT-PCR of RNA extracted from infected 293T or 293T ACE2 TMPRSS2 cells in A. Arrow indicates expected band size. (C) RT-qPCR analysis of samples in (B). (D) Spike RBD RT-PCR of RNA extracted from infected 293T ACE2 TMPRSS2 cells with VLPs generated from 293T Spike PS9 cells transfected with indicated amounts of structural protein expression vectors. (E) RT-qPCR analysis of samples in (D). (F) Spike RBD RT-qPCR analysis of RNA from 293T ACE2 TMPRSS2 cells infected with VLPs generated from 293T Spike PS9 cells. The VLPs were used either unconcentrated (“Sup”) or were concentrated over 20% sucrose cushion and then used for infection (“Pellet”).

FIGS. 2A-2B shows a virus-like particle platform for deep mutational scanning of the SARS-CoV-2 Spike protein. (A) The approach to deep mutational scanning of the SARS-CoV-2 Spike protein. The Spike coding sequence was cloned into a lentiviral vector backbone containing Puromycin resistance and the SARS-CoV-2 packaging signal PS9. The Spike RBD was mutagenized at every position with all possible 20 aa. The library was transduced into 293T cells and transfected with expression vectors for the Membrane, Envelope, and Nucleocapsid proteins. VLPs were collected and used to infect 293T ACE2 TMPRSS2 cells. RNA was collected from producer cells, VLPs, and infected cells for RT-PCR and long-read PacBio sequencing across the RBD. (B) Distribution of types of mutation across all four replicas of the deep mutational scan. (C) Distribution of Producer cell, VLP, and infected cell counts per number of unique variants.

FIGS. 3A-3H demonstrate that the Spike RBD deep mutational scan reveals mutational constraints on viral particle assembly. (A) and (B) Heatmaps of VLP production and entry measurements as log enrichment of counts in VLPs and recipient cells, respectively, relative to counts in producer cells for mutations across the Spike RBD. White banded squares indicate mutations that were not observed in VLPs or recipient cells. (C) Distribution of mean VLP production values across the Spike RBD. The wildtype VLP production value is indicated by the red star. (D) Distribution of VLP entry values across the Spike RBD The wildtype VLP entry value is indicated by the red star. (E) Distribution of Producer cell counts of all mutations across the Spike RBD vs mutations that were associated with low (≤10) or high (≥500) counts in VLPs (F) The variability of the VLP entry data is depicted as the standard deviation and mean VLP entry values across four biological replicates. (G) VLP entry as a function of VLP production for each single mutant; significant measurements for both assembly and infectivity are denoted by a deeper color. The wildtype is indicated by the red star. (H) Correlation of the VLP entry dataset with yeast surface display RBD expression and ACE2 binding (data adapted from 10.1016/j.cell.2020.08.012) as well as PTV entry (data adapted from doi.org/10.1101/2023.11.13.566961). Spearman's r for each correlation is indicated on top of each graph.

FIGS. 4A-4D shows that Spike RBD mutations impact loading of Spike onto VLPs. (A) Schematic of validation experiment. (B) Western blot analysis of the abundance of the Spike and Nucleocapsid proteins in cell lysate and VLPs. GAPDH and P24 were included as loading internal controls for cell lysate and VLPs, respectively. The indicated mutants were chosen because they exhibited significantly increased or decreased production and entry (indicated with red and green arrows). Those with statistically insignificant results were indicated with a black dash. (C) Correlation between Spike assembly onto particle as measured by Western blot (x-axis) and DMS VLP production (y-axis). Non-bald particles are indicated in blue dots and bald particles are indicated in orange dots. (D) Correlation between VLP entry as measured by VLP infection assay (x-axis) and DMS VLP entry (y-axis). Non-bald particles are indicated in blue dots and bald particles are indicated in orange dots.

FIGS. 5A-5C demonstrate that Spike RBD mutations impact Spike processing. (A) Schematic of the experiment to characterize Spike variant processing. (B) Western blot analysis of the abundance of Spike in cell lysate with and without digestion with PNGaseF and EndoH enzymes. GAPDH was included as an internal loading control. The arrows indicate the different bands observed for the Spike protein pre- and post-digestion. (C) Immunofluorescence analysis of indicated Spike mutants, Nucleocapsid, and GRP78 (ER marker) proteins in VLP-producing cells. UT indicates untransfected control. Scale bar=10 μm.

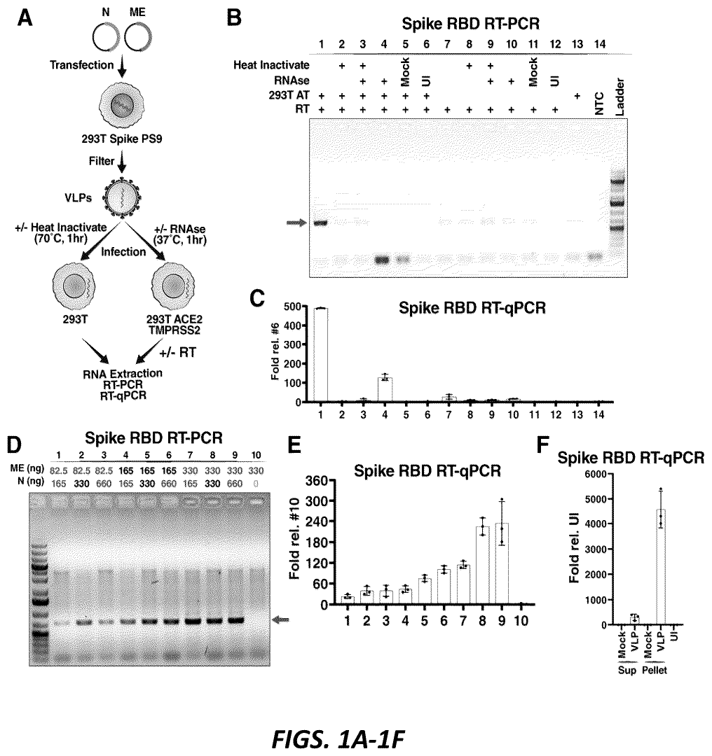

SUPP. FIGS. 1A-1B demonstrate that SARS-CoV-2 virus-like particles can be generated in cells transfected or transduced with Spike expression vector. (A) VLPs were generated under standard conditions (S+E+M+N) or without Spike (E+M+N) and Nucleocapsid (E+M) expression vectors in untransduced or Spike-transduced 293T cells. Luciferase readout in infected 293T ACE2 TMPRSS2 cells was used as a readout for VLP assembly and entry. (B) VLPs were generated without Spike expression vector in untransduced, Spike-, or Spike PS9-transduced 293T cells. Luciferase readout in infected 293T ACE2 TMPRSS2 cells was used as a readout for VLP assembly and entry.

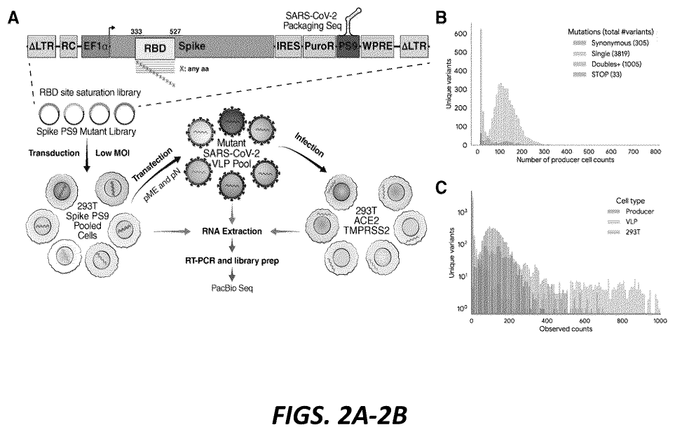

SUPP. FIGS. 2A-2C provide Optimization of the generation of SARS-CoV-2 virus-like particles packaging Spike PS9 transcript. (A) Schematic of experiment to optimize collection time of VLPs packaging Spike PS9 transcript post-transfection. (B) Spike RBD RT-PCR of RNA from infected 293T ACE2 TMPRSS2 cells. Red arrow indicates expected band size. (C) RT-qPCR analysis of samples in B. hpt: hours post-transfection; UI: uninfected; NTC: no template control.

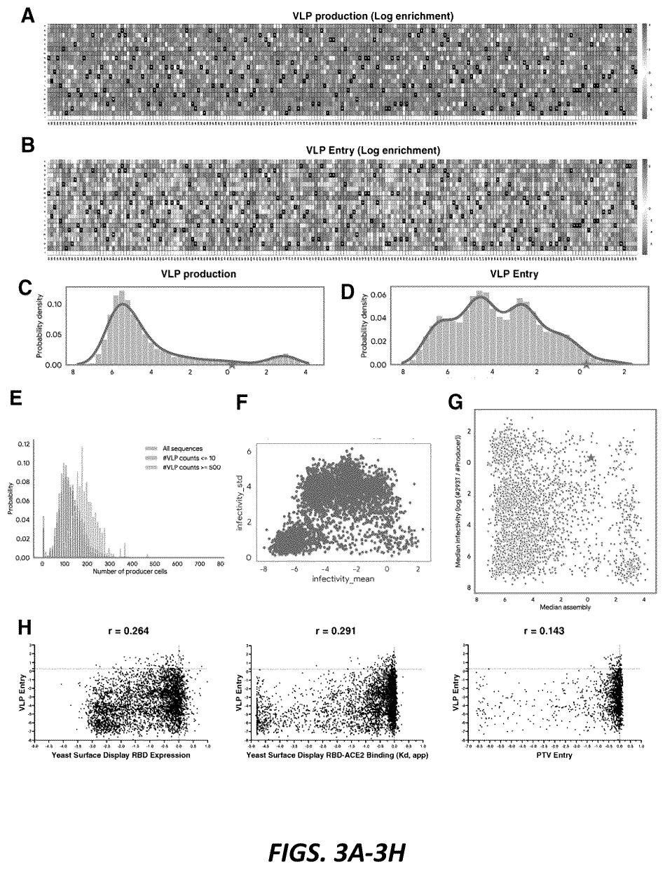

SUPP. FIG. 3 shows sequencing output overview for all experiments. The raw, filtered and trimmed, and mapped PacBio reads are plotted for each of the four experiments.

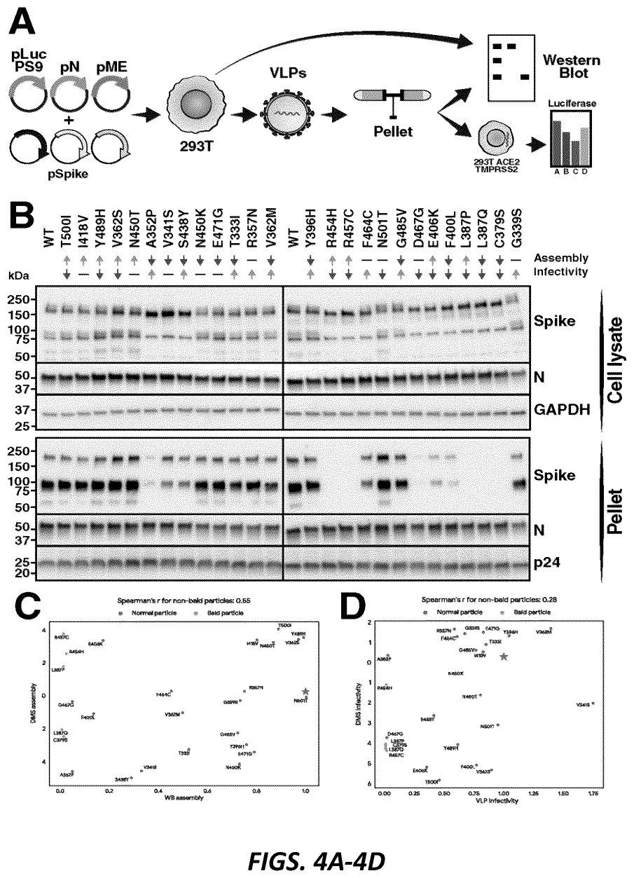

SUPP. FIG. 4 provides producer cell counts of deep mutational scan mutants of the SARS-CoV-2 Spike RBD. The abundance of each mutation across the RBD is depicted on a heatmap.

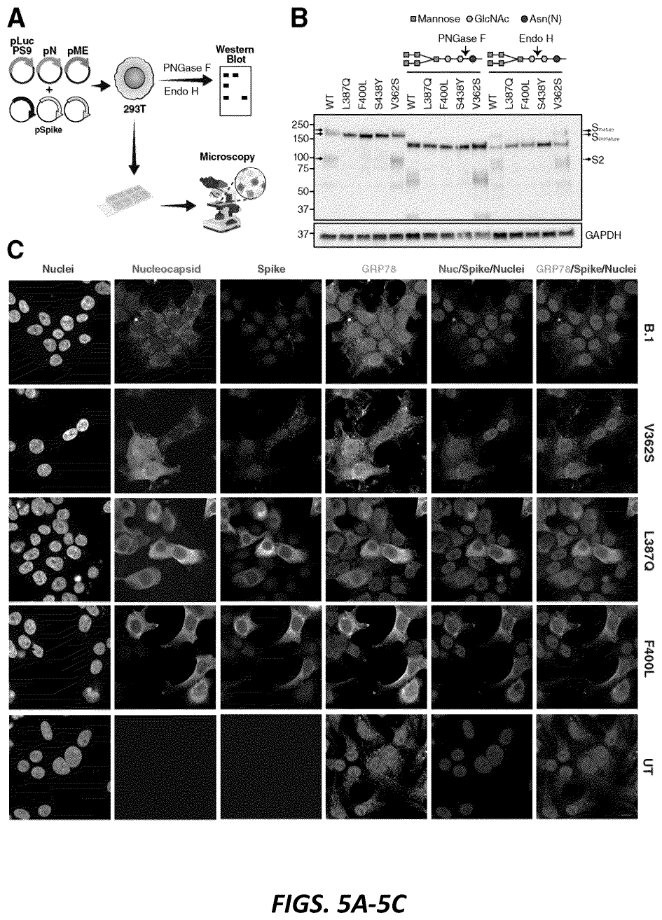

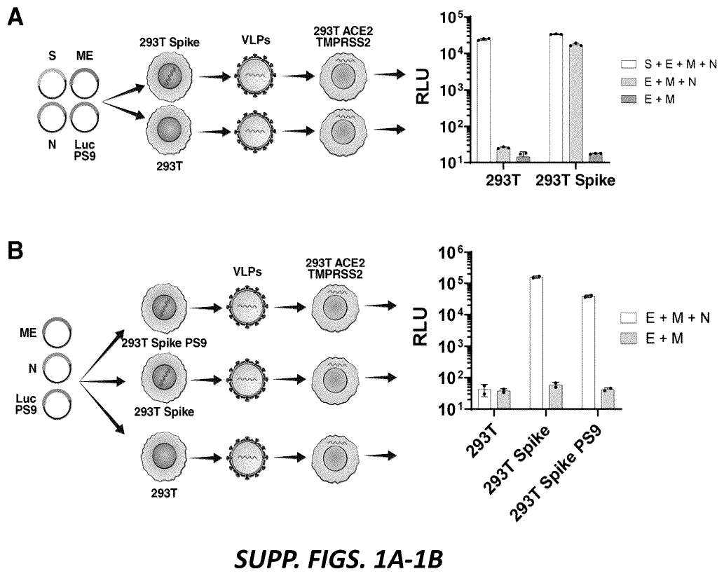

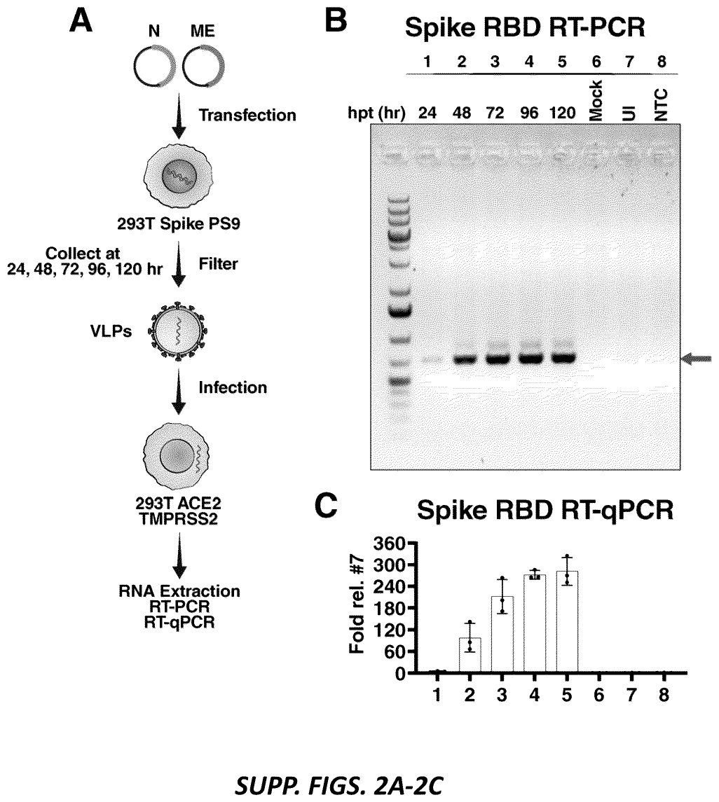

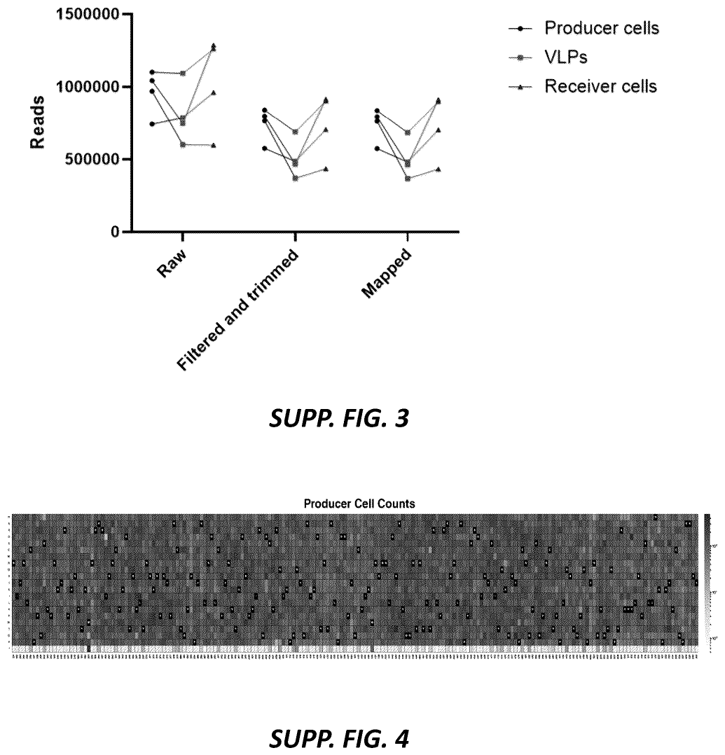

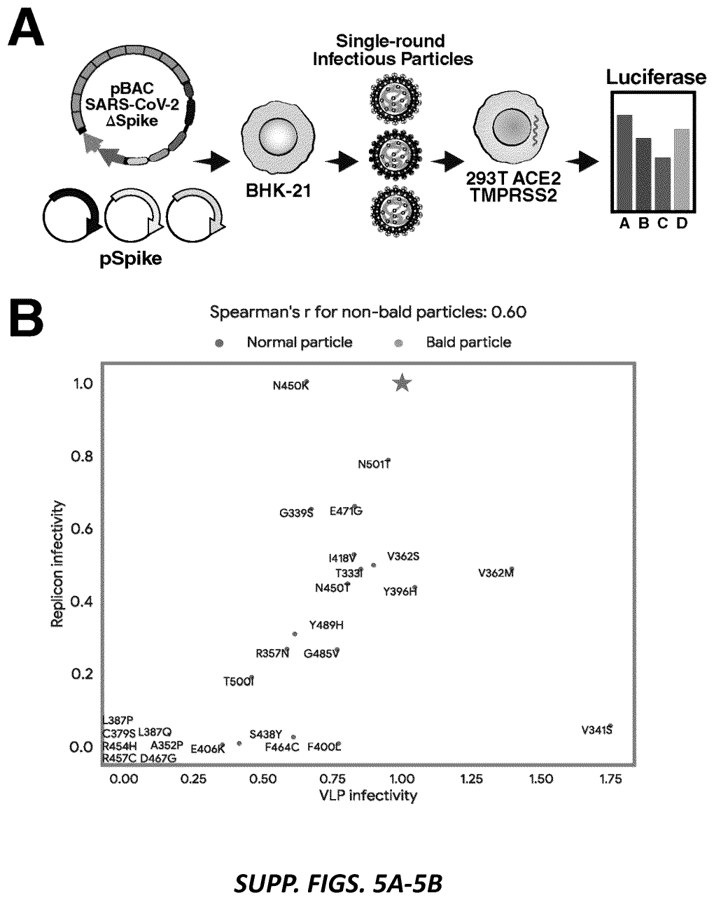

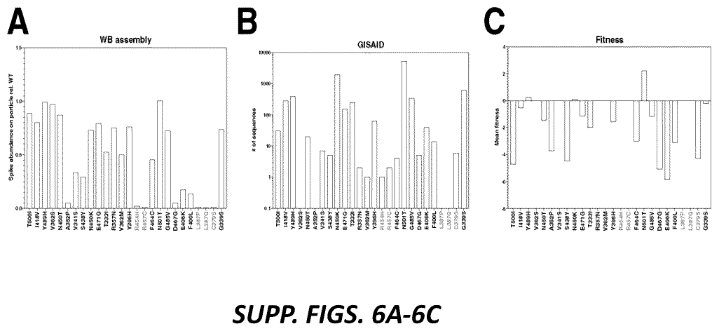

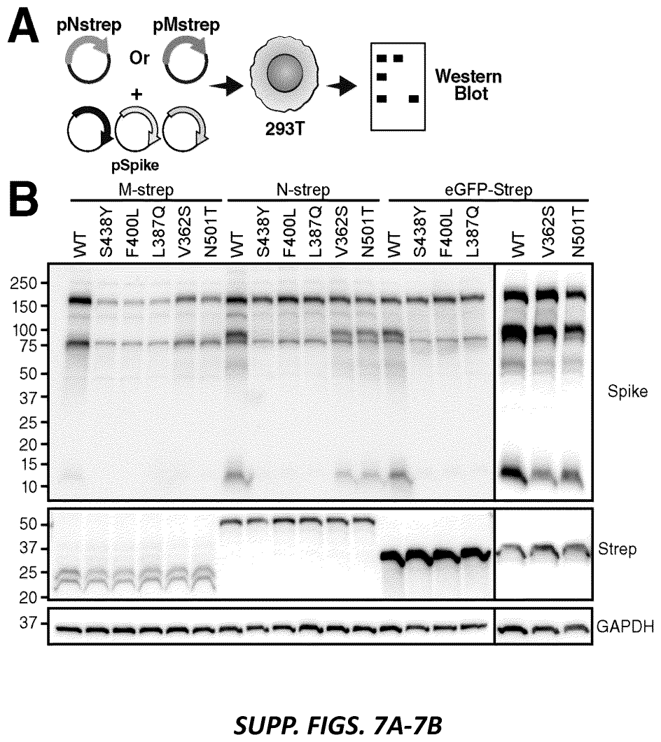

SUPP. FIGS. 5A-5B demonstrates that VLP infectivity measurement correlates with Spike replicon infectivity measurement. (A) Schematic of Spike replicon infectivity measurement. (B) Correlation of Replicon and VLP infectivity measurements for indicated mutants. VLP infection data is from the experiment in FIG. 4. Bald particles are indicated in orange dots and non-bald particles indicated in blue dots. The wild-type Spike (D614G) is indicated in a red star.

SUPP. FIGS. 6A-6C provide characteristics of Spike mutants in validation experiment in FIG. 4. (A) The experimental measurement of Spike abundance on VLPs from the experiment in FIG. 4. (B) The abundance of sequences containing indicated mutations in the GISAID database. Only genomes >29,000 nt and <5% Ns (undefined bases) were included. (C) The fitness of indicated mutations were obtained from a recently published fitness effects calculation by Bloom et al.

SUPP. FIGS. 7A-7B demonstrates that the SARS-CoV-2 M protein reduces Spike processing. (A) Schematic of experiment to determine the role of structural proteins N and M on Spike processing. (B) Western blot analysis of Spike and strep-tagged M, N, or eGFP in cells expressing indicated structural proteins with the Spike protein. GAPDH was utilized as an internal loading control.

### DESCRIPTION OF THE INVENTION

The continued emergence of SARS-CoV-2 viral variants represents a lingering threat to our efforts to curb the COVID-19 pandemic. To proactively adjust vaccines and therapeutics, an in-depth understanding of the mutational constraints of viral evolution is needed A high-throughput virus-like particle (VLP) platform was developed, faithfully recapitulating viral entry and assembly steps, to conduct a deep mutational scan (˜3.8K mutants) of the Spike receptor binding domain (RBD). It was found that the majority of mutants across the RBD negatively impact assembly of Spike onto the viral particle due to decreased Spike glycosylation and maturation. VLP-based deep mutational scanning adds critical insight to the molecular understanding of SARS-CoV-2 evolution and uncovers an unexpected role of the Spike RBD in particle assembly.

The invention provides a high-throughput screening of SARS-CoV-2 variants, that allows for one to, for example, screen low-prevalence currently circulating SARS-CoV-2 variants for enhanced infectivity/neutralization escape, identify future SARS-CoV-2 variants with enhanced infectivity/neutralization escape and create future, preventive SARS-CoV-2 vaccines (vaccine strain selection) and/or immunomodulating compounds.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N. Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

SARS-CoV-2 is a member of a large family of viruses called coronaviruses. These viruses can infect people and some animals. SARS-CoV-2 was first known to infect people in 2019. The virus is thought to spread from person to person through droplets released when an infected person coughs, sneezes, or talks.

Like other coronaviruses, SARS-CoV-2 has four structural proteins, known as the S (Spike), E (envelope), M (membrane), and N (nucleocapsid) proteins; the N protein holds the RNA genome, and the S, E, and M proteins together create the viral envelope. Coronavirus S proteins are glycoproteins and also type I membrane proteins (membranes containing a single transmembrane domain oriented on the extracellular side). They are divided into two functional parts (S1 and S2). In SARS-CoV-2, the Spike protein is the protein responsible for allowing the virus to attach to and fuse with the membrane of a host cell; specifically, its S1 subunit catalyzes attachment, the S2 subunit fusion.

### Mutant Libraries

The study of proteins is extremely beneficial in relation to viruses, such as SARS-CoV-2. Many viruses can be effectively managed or treated. For example, vaccination has all but ameliorated smallpox and measles, once among mankind's greatest scourges. Unfortunately, however, numerous viruses continue to pose significant health threats. Examples include influenza virus, human immunodeficiency virus (HIV), Ebola virus, Middle Eastern respiratory syndrome coronavirus (MERS-CoV) and SAR-CoV-2.

To combat the spread of viruses, scientists and doctors need tools to know when drugs, vaccines, or antibodies are effectively working against viral proteins, or conversely, when these viral proteins have developed resistance to these countermeasures and pose a greater risk.

Viral entry proteins are a primary target of immune system responses against viral infections. Most vaccines elicit neutralizing antibodies to the viral entry protein. Therapeutic antibodies can also be used to impair the activity of viral entry proteins, with the potential to both protect against infection as well as to therapeutically treat active infection. However, viral entry proteins are able to mutate and evolve over time, and mutations can allow these proteins to escape recognition by immune system responses and therapeutic antibodies. Evasion or susceptibility to neutralization by antibodies can be examined using mutant viral entry proteins in antibody neutralization assays.

Mutagenesis refers to altering the amino acid that naturally occurs at a position along the string of amino acids that creates a given protein. Systematically altering amino acids at different positions through mutagenesis can identify those amino acids that are essential to the function of the protein. Deep mutational scanning refers to methods of generating and characterizing hundreds of thousands of mutants or more of a given protein. More particularly, deep mutational scanning can refer to altering each amino acid position with all possible alternative amino acids.

A library of mutant Spike proteins can be generated by methods available to an art worker (or other structural proteins, such as M, N and/or E). For example, in one embodiment, PCR mutagenesis is used. PCR mutagenesis is a method for generating site-directed mutagenesis. This method can generate mutations (base substitutions, insertions, deletions chimeric gene generation, multiple-site mutagenesis, and random mutagenesis at either a single site or multiple sites). For example, by using a using a pair of oligonucleotide primers designed with mismatching nucleotides in the primers. Numerous PCR-based methods have been developed commercially or noncommercially. Among those methods, the overlap extension method (Higuchi R, et al. Nucleic Acids Res 16: 7351-7367, 1988), megaprimer method (Kammann M, et al. Nucleic Acids Res 17: 5404, 1989), Quick Change Method (Stratagene, La Jolla, CA), and their modified versions (Ke S H and Madison E L. Nucleic Acids Res. 25: 3371-3372, 1997; Urban A, et al. Nucleic Acids Res. 25: 2227-2228, 1997; Zheng L, et al Nucleic Acids Res 32:e115, 2004) are examples. In another embodiment, Site-directed Mutagenesis for Large Plasmids (SMLP) (Zhang et al. Scientific Reports volume 11, Article number: 10454 (2021)) can be used.

In one embodiment, mutations/mutant libraries of SARS-CoV-2 structural proteins (S, M, N, and E) can be generated by PCR mutagenesis with a primer containing the desired mutations. In other embodiments, mutant libraries are generated by synthesis (see, for example, Twist Bioscience; twistbioscience.com/products/libraries/site-saturation-libraries). One, two, three or more codon mutations can be generated in each structural protein. One or more of these mutated sequences are then used to create the VLP discussed below (S, M, N, E and a cis-acting RNA sequence that triggers packaging).

This allows the production of thousands of variants that can be studied in a single pooled experiment. PacBio sequencing, or other methods available to an art worker, can be used in the methods discussed herein.

Provided herein is a high-throughput approach to screen SARS-CoV-2 Spike variants in the context of the full Spike protein. The SARS-CoV-2 RNA packaging signal, termed packaging signal 9 (PS9) was used to enable incorporation of Spike expression transcript into virus-like particles (VLPs). Therefore, cells transduced with an expression construct of Spike-PS9 and later on transfected with expression constructs of the other viral structural proteins will assemble SARS-CoV-2 VLPs decorated with a specific Spike variant but also delivering the sequence of that Spike variant into receiver cells. Using this concept, the method was then optimized for pooled screening of Spike variants.

A library of Spike variants was transduced into 293T cells at a low MOI. Cells were expanded and transfected with plasmids expressing the SARS-CoV-2 E, M, and N proteins. Supernatants will be used to infect 293T ACE2 TMPRSS2 cells. To assess neutralization escape, the VLP pool can be pre-incubated with vaccine sera before infection. RNA can be extracted from the VLP pool and infected cells and PacBio sequenced to determine enrichment of each Spike variant.

As a proof-of-concept, a library of Spike variants focused at the 3′ end of the RBD was synthesized and it was shown that some variants improve infectivity, and some do not. Indeed, some well-characterized mutations (e.g., N501Y) significantly improved infectivity, whereas other positions (e.g., 495 and 497) were less tolerant of mutations. However, some positions did not completely agree with published deep mutational scanning data, thereby suggesting interesting differences in testing Spike mutations in the context of the entire Spike sequence as opposed to the RBD alone.

An example of SARS-CoV-2 Spike protein that can be used in the methods of invention, such as to create a mutant library, has the sequence of (NCBI Reference Sequence: YP_009724390.1):

surface glycoprotein [severe acute respiratory
syndrome coronavirus 2]
(SEQ ID NO: 1)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVISFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

An example of SARS-CoV-2 E protein that can be used in the methods of invention, such as to create a mutant library, has the sequence of (NCBI Reference Sequence: YP_009724392.1):

>YP_009724392.1 envelope protein [severe acute
respiratory syndrome coronavirus 2]
(SEQ ID NO: 2)
MYSFVSEETGTLIVNSVLLFLAFVVFLLVTLAILTALRICAYCCNIVNVS
LVKPSFYVYSRVKNLNSSRVPDLLV

An example of SARS-CoV-2 M protein that can be used in the methods of invention, such as to create a mutant library, has the sequence of (NCBI Reference Sequence: YP_009724393.1):

>YP_009724393.1 membrane glycoprotein [severe
acute respiratory syndrome coronavirus 2]
(SEQ ID NO: 3)
MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHER
IAGHHLGRCDIKDLPKEITVATSRTLSYYKLGASQRVAGDSGFAAYSRYR
IGNYKLNTDHSSSSDNIALLVQ

An example of SARS-CoV-2 N protein that can be used in the methods of invention, such as to create a mutant library, has the sequence of (NCBI Reference Sequence: YP 009724397.2):

>YP_009724397.2 nucleocapsid phosphoprotein
[severe acute respiratory syndrome coronavirus 2]
(SEQ ID NO: 4)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRINQLESKMSGKGQQQQGQTVTKKS
AAEASKKPROKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA

### Production of VLPs

A process that mimics viral assembly to package and deliver reporter transcripts would simplify the analysis of successful virus production, budding, and entry. Previous studies have shown that coexpression of only the structural proteins of coronaviruses generates virus like particles (VLPs) that contain all four structural proteins (12-17). These VLPs appear to have similar morphology to infectious viruses.

A requirement for such VLPs to deliver reporter transcripts into cells is the recognition of a cis-acting RNA sequence that triggers packaging. Provided herein the packaging signal sequences can include, for example, 200 to 1500 nucleotides of T20 (nucleotides 20080 to 22222) located near the 3′ end of ORG1ab of SARS-CoV-2. For example, in one embodiment, the packaging signal is PS9 (nucleotides 20080 to 21171), as an exemplary packaging signal (Syed et al., Science 374, 1626-1632 (2021)). However, as will be understood by one of ordinary skill in the art, a packaging signal can refer to the shortest sequence required to allow packaging of viral material. In particular embodiments, the packaging signal includes 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, or 200 or more nucleotides of T20 (nucleotides 20080 to 22222.

The numbering above for the PS9 and T20 sequence correlate to NCBI Reference Sequence: NC_045512.2:

>NC_045512.2 severe acute respiratory syndrome coronavirus 2 isolate Wuhan-
Hu-1, complete genome
(SEQ ID NO: 5)
ATTAAAGGTTTATACCTTCCCAGGTAACAAACCAACCAACTTTCGATCTCTTGTAGATCTGTTCTCTAAA
CGAACTTTAAAATCTGTGTGGCTGTCACTCGGCTGCATGCTTAGTGCACTCACGCAGTATAATTAATAAC
TAATTACTGTCGTTGACAGGACACGAGTAACTCGTCTATCTTCTGCAGGCTGCTTACGGTTTCGTCCGTG
TTGCAGCCGATCATCAGCACATCTAGGTTTCGTCCGGGTGTGACCGAAAGGTAAGATGGAGAGCCTTGTC
CCTGGTTTCAACGAGAAAACACACGTCCAACTCAGTTTGCCTGTTTTACAGGTTCGCGACGTGCTCGTAC
GTGGCTTTGGAGACTCCGTGGAGGAGGTCTTATCAGAGGCACGTCAACATCTTAAAGATGGCACTTGTGG
CTTAGTAGAAGTTGAAAAAGGCGTTTTGCCTCAACTTGAACAGCCCTATGTGTTCATCAAACGTTCGGAT
GCTCGAACTGCACCTCATGGTCATGTTATGGTTGAGCTGGTAGCAGAACTCGAAGGCATTCAGTACGGTC
GTAGTGGTGAGACACTTGGTGTCCTTGTCCCTCATGTGGGCGAAATACCAGTGGCTTACCGCAAGGTTCT
TCTTCGTAAGAACGGTAATAAAGGAGCTGGTGGCCATAGTTACGGCGCCGATCTAAAGTCATTTGACTTA
GGCGACGAGCTTGGCACTGATCCTTATGAAGATTTTCAAGAAAACTGGAACACTAAACATAGCAGTGGTG
TTACCCGTGAACTCATGCGTGAGCTTAACGGAGGGGCATACACTCGCTATGTCGATAACAACTTCTGTGG
CCCTGATGGCTACCCTCTTGAGTGCATTAAAGACCTTCTAGCACGTGCTGGTAAAGCTTCATGCACTTTG
TCCGAACAACTGGACTTTATTGACACTAAGAGGGGTGTATACTGCTGCCGTGAACATGAGCATGAAATTG
CTTGGTACACGGAACGTTCTGAAAAGAGCTATGAATTGCAGACACCTTTTGAAATTAAATTGGCAAAGAA
ATTTGACACCTTCAATGGGGAATGTCCAAATTTTGTATTTCCCTTAAATTCCATAATCAAGACTATTCAA
CCAAGGGTTGAAAAGAAAAAGCTTGATGGCTTTATGGGTAGAATTCGATCTGTCTATCCAGTTGCGTCAC
CAAATGAATGCAACCAAATGTGCCTTTCAACTCTCATGAAGTGTGATCATTGTGGTGAAACTTCATGGCA
GACGGGCGATTTTGTTAAAGCCACTTGCGAATTTTGTGGCACTGAGAATTTGACTAAAGAAGGTGCCACT
ACTTGTGGTTACTTACCCCAAAATGCTGTTGTTAAAATTTATTGTCCAGCATGTCACAATTCAGAAGTAG
GACCTGAGCATAGTCTTGCCGAATACCATAATGAATCTGGCTTGAAAACCATTCTTCGTAAGGGTGGTCG
CACTATTGCCTTTGGAGGCTGTGTGTTCTCTTATGTTGGTTGCCATAACAAGTGTGCCTATTGGGTTCCA
CGTGCTAGCGCTAACATAGGTTGTAACCATACAGGTGTTGTTGGAGAAGGTTCCGAAGGTCTTAATGACA
ACCTTCTTGAAATACTCCAAAAAGAGAAAGTCAACATCAATATTGTTGGTGACTTTAAACTTAATGAAGA
GATCGCCATTATTTTGGCATCTTTTTCTGCTTCCACAAGTGCTTTTGTGGAAACTGTGAAAGGTTTGGAT
TATAAAGCATTCAAACAAATTGTTGAATCCTGTGGTAATTTTAAAGTTACAAAAGGAAAAGCTAAAAAAG
GTGCCTGGAATATTGGTGAACAGAAATCAATACTGAGTCCTCTTTATGCATTTGCATCAGAGGCTGCTCG
TGTTGTACGATCAATTTTCTCCCGCACTCTTGAAACTGCTCAAAATTCTGTGCGTGTTTTACAGAAGGCC
GCTATAACAATACTAGATGGAATTTCACAGTATTCACTGAGACTCATTGATGCTATGATGTTCACATCTG
ATTTGGCTACTAACAATCTAGTTGTAATGGCCTACATTACAGGTGGTGTTGTTCAGTTGACTTCGCAGTG
GCTAACTAACATCTTTGGCACTGTTTATGAAAAACTCAAACCCGTCCTTGATTGGCTTGAAGAGAAGTTT
AAGGAAGGTGTAGAGTTTCTTAGAGACGGTTGGGAAATTGTTAAATTTATCTCAACCTGTGCTTGTGAAA
TTGTCGGTGGACAAATTGTCACCTGTGCAAAGGAAATTAAGGAGAGTGTTCAGACATTCTTTAAGCTTGT
AAATAAATTTTTGGCTTTGTGTGCTGACTCTATCATTATTGGTGGAGCTAAACTTAAAGCCTTGAATTTA
GGTGAAACATTTGTCACGCACTCAAAGGGATTGTACAGAAAGTGTGTTAAATCCAGAGAAGAAACTGGCC
TACTCATGCCTCTAAAAGCCCCAAAAGAAATTATCTTCTTAGAGGGAGAAACACTTCCCACAGAAGTGTT
AACAGAGGAAGTTGTCTTGAAAACTGGTGATTTACAACCATTAGAACAACCTACTAGTGAAGCTGTTGAA
GCTCCATTGGTTGGTACACCAGTTTGTATTAACGGGCTTATGTTGCTCGAAATCAAAGACACAGAAAAGT
ACTGTGCCCTTGCACCTAATATGATGGTAACAAACAATACCTTCACACTCAAAGGCGGTGCACCAACAAA
GGTTACTTTTGGTGATGACACTGTGATAGAAGTGCAAGGTTACAAGAGTGTGAATATCACTTTTGAACTT
GATGAAAGGATTGATAAAGTACTTAATGAGAAGTGCTCTGCCTATACAGTTGAACTCGGTACAGAAGTAA
ATGAGTTCGCCTGTGTTGTGGCAGATGCTGTCATAAAAACTTTGCAACCAGTATCTGAATTACTTACACC
ACTGGGCATTGATTTAGATGAGTGGAGTATGGCTACATACTACTTATTTGATGAGTCTGGTGAGTTTAAA
TTGGCTTCACATATGTATTGTTCTTTCTACCCTCCAGATGAGGATGAAGAAGAAGGTGATTGTGAAGAAG
AAGAGTTTGAGCCATCAACTCAATATGAGTATGGTACTGAAGATGATTACCAAGGTAAACCTTTGGAATT
TGGTGCCACTTCTGCTGCTCTTCAACCTGAAGAAGAGCAAGAAGAAGATTGGTTAGATGATGATAGTCAA
CAAACTGTTGGTCAACAAGACGGCAGTGAGGACAATCAGACAACTACTATTCAAACAATTGTTGAGGTTC
AACCTCAATTAGAGATGGAACTTACACCAGTTGTTCAGACTATTGAAGTGAATAGTTTTAGTGGTTATTT
AAAACTTACTGACAATGTATACATTAAAAATGCAGACATTGTGGAAGAAGCTAAAAAGGTAAAACCAACA
GTGGTTGTTAATGCAGCCAATGTTTACCTTAAACATGGAGGAGGTGTTGCAGGAGCCTTAAATAAGGCTA
CTAACAATGCCATGCAAGTTGAATCTGATGATTACATAGCTACTAATGGACCACTTAAAGTGGGTGGTAG
TTGTGTTTTAAGCGGACACAATCTTGCTAAACACTGTCTTCATGTTGTCGGCCCAAATGTTAACAAAGGT
GAAGACATTCAACTTCTTAAGAGTGCTTATGAAAATTTTAATCAGCACGAAGTTCTACTTGCACCATTAT
TATCAGCTGGTATTTTTGGTGCTGACCCTATACATTCTTTAAGAGTTTGTGTAGATACTGTTCGCACAAA
TGTCTACTTAGCTGTCTTTGATAAAAATCTCTATGACAAACTTGTTTCAAGCTTTTTGGAAATGAAGAGT
GAAAAGCAAGTTGAACAAAAGATCGCTGAGATTCCTAAAGAGGAAGTTAAGCCATTTATAACTGAAAGTA
AACCTTCAGTTGAACAGAGAAAACAAGATGATAAGAAAATCAAAGCTTGTGTTGAAGAAGTTACAACAAC
TCTGGAAGAAACTAAGTTCCTCACAGAAAACTTGTTACTTTATATTGACATTAATGGCAATCTTCATCCA
GATTCTGCCACTCTTGTTAGTGACATTGACATCACTTTCTTAAAGAAAGATGCTCCATATATAGTGGGTG
ATGTTGTTCAAGAGGGTGTTTTAACTGCTGTGGTTATACCTACTAAAAAGGCTGGTGGCACTACTGAAAT
GCTAGCGAAAGCTTTGAGAAAAGTGCCAACAGACAATTATATAACCACTTACCCGGGTCAGGGTTTAAAT
GGTTACACTGTAGAGGAGGCAAAGACAGTGCTTAAAAAGTGTAAAAGTGCCTTTTACATTCTACCATCTA
TTATCTCTAATGAGAAGCAAGAAATTCTTGGAACTGTTTCTTGGAATTTGCGAGAAATGCTTGCACATGC
AGAAGAAACACGCAAATTAATGCCTGTCTGTGTGGAAACTAAAGCCATAGTTTCAACTATACAGCGTAAA
TATAAGGGTATTAAAATACAAGAGGGTGTGGTTGATTATGGTGCTAGATTTTACTTTTACACCAGTAAAA
CAACTGTAGCGTCACTTATCAACACACTTAACGATCTAAATGAAACTCTTGTTACAATGCCACTTGGCTA
TGTAACACATGGCTTAAATTTGGAAGAAGCTGCTCGGTATATGAGATCTCTCAAAGTGCCAGCTACAGTT
TCTGTTTCTTCACCTGATGCTGTTACAGCGTATAATGGTTATCTTACTTCTTCTTCTAAAACACCTGAAG
AACATTTTATTGAAACCATCTCACTTGCTGGTTCCTATAAAGATTGGTCCTATTCTGGACAATCTACACA
ACTAGGTATAGAATTTCTTAAGAGAGGTGATAAAAGTGTATATTACACTAGTAATCCTACCACATTCCAC
CTAGATGGTGAAGTTATCACCTTTGACAATCTTAAGACACTTCTTTCTTTGAGAGAAGTGAGGACTATTA
AGGTGTTTACAACAGTAGACAACATTAACCTCCACACGCAAGTTGTGGACATGTCAATGACATATGGACA
ACAGTTTGGTCCAACTTATTTGGATGGAGCTGATGTTACTAAAATAAAACCTCATAATTCACATGAAGGT
AAAACATTTTATGTTTTACCTAATGATGACACTCTACGTGTTGAGGCTTTTGAGTACTACCACACAACTG
ATCCTAGTTTTCTGGGTAGGTACATGTCAGCATTAAATCACACTAAAAAGTGGAAATACCCACAAGTTAA
TGGTTTAACTTCTATTAAATGGGCAGATAACAACTGTTATCTTGCCACTGCATTGTTAACACTCCAACAA
ATAGAGTTGAAGTTTAATCCACCTGCTCTACAAGATGCTTATTACAGAGCAAGGGCTGGTGAAGCTGCTA
ACTTTTGTGCACTTATCTTAGCCTACTGTAATAAGACAGTAGGTGAGTTAGGTGATGTTAGAGAAACAAT
GAGTTACTTGTTTCAACATGCCAATTTAGATTCTTGCAAAAGAGTCTTGAACGTGGTGTGTAAAACTTGT
GGACAACAGCAGACAACCCTTAAGGGTGTAGAAGCTGTTATGTACATGGGCACACTTTCTTATGAACAAT
TTAAGAAAGGTGTTCAGATACCTTGTACGTGTGGTAAACAAGCTACAAAATATCTAGTACAACAGGAGTC
ACCTTTTGTTATGATGTCAGCACCACCTGCTCAGTATGAACTTAAGCATGGTACATTTACTTGTGCTAGT
GAGTACACTGGTAATTACCAGTGTGGTCACTATAAACATATAACTTCTAAAGAAACTTTGTATTGCATAG
ACGGTGCTTTACTTACAAAGTCCTCAGAATACAAAGGTCCTATTACGGATGTTTTCTACAAAGAAAACAG
TTACACAACAACCATAAAACCAGTTACTTATAAATTGGATGGTGTTGTTTGTACAGAAATTGACCCTAAG
TTGGACAATTATTATAAGAAAGACAATTCTTATTTCACAGAGCAACCAATTGATCTTGTACCAAACCAAC
CATATCCAAACGCAAGCTTCGATAATTTTAAGTTTGTATGTGATAATATCAAATTTGCTGATGATTTAAA
CCAGTTAACTGGTTATAAGAAACCTGCTTCAAGAGAGCTTAAAGTTACATTTTTCCCTGACTTAAATGGT
GATGTGGTGGCTATTGATTATAAACACTACACACCCTCTTTTAAGAAAGGAGCTAAATTGTTACATAAAC
CTATTGTTTGGCATGTTAACAATGCAACTAATAAAGCCACGTATAAACCAAATACCTGGTGTATACGTTG
TCTTTGGAGCACAAAACCAGTTGAAACATCAAATTCGTTTGATGTACTGAAGTCAGAGGACGCGCAGGGA
ATGGATAATCTTGCCTGCGAAGATCTAAAACCAGTCTCTGAAGAAGTAGTGGAAAATCCTACCATACAGA
AAGACGTTCTTGAGTGTAATGTGAAAACTACCGAAGTTGTAGGAGACATTATACTTAAACCAGCAAATAA
TAGTTTAAAAATTACAGAAGAGGTTGGCCACACAGATCTAATGGCTGCTTATGTAGACAATTCTAGTCTT
ACTATTAAGAAACCTAATGAATTATCTAGAGTATTAGGTTTGAAAACCCTTGCTACTCATGGTTTAGCTG
CTGTTAATAGTGTCCCTTGGGATACTATAGCTAATTATGCTAAGCCTTTTCTTAACAAAGTTGTTAGTAC
AACTACTAACATAGTTACACGGTGTTTAAACCGTGTTTGTACTAATTATATGCCTTATTTCTTTACTTTA
TTGCTACAATTGTGTACTTTTACTAGAAGTACAAATTCTAGAATTAAAGCATCTATGCCGACTACTATAG
CAAAGAATACTGTTAAGAGTGTCGGTAAATTTTGTCTAGAGGCTTCATTTAATTATTTGAAGTCACCTAA
TTTTTCTAAACTGATAAATATTATAATTTGGTTTTTACTATTAAGTGTTTGCCTAGGTTCTTTAATCTAC
TCAACCGCTGCTTTAGGTGTTTTAATGTCTAATTTAGGCATGCCTTCTTACTGTACTGGTTACAGAGAAG
GCTATTTGAACTCTACTAATGTCACTATTGCAACCTACTGTACTGGTTCTATACCTTGTAGTGTTTGTCT
TAGTGGTTTAGATTCTTTAGACACCTATCCTTCTTTAGAAACTATACAAATTACCATTTCATCTTTTAAA
TGGGATTTAACTGCTTTTGGCTTAGTTGCAGAGTGGTTTTTGGCATATATTCTTTTCACTAGGTTTTTCT
ATGTACTTGGATTGGCTGCAATCATGCAATTGTTTTTCAGCTATTTTGCAGTACATTTTATTAGTAATTC
TTGGCTTATGTGGTTAATAATTAATCTTGTACAAATGGCCCCGATTTCAGCTATGGTTAGAATGTACATC
TTCTTTGCATCATTTTATTATGTATGGAAAAGTTATGTGCATGTTGTAGACGGTTGTAATTCATCAACTT
GTATGATGTGTTACAAACGTAATAGAGCAACAAGAGTCGAATGTACAACTATTGTTAATGGTGTTAGAAG
GTCCTTTTATGTCTATGCTAATGGAGGTAAAGGCTTTTGCAAACTACACAATTGGAATTGTGTTAATTGT
GATACATTCTGTGCTGGTAGTACATTTATTAGTGATGAAGTTGCGAGAGACTTGTCACTACAGTTTAAAA
GACCAATAAATCCTACTGACCAGTCTTCTTACATCGTTGATAGTGTTACAGTGAAGAATGGTTCCATCCA
TCTTTACTTTGATAAAGCTGGTCAAAAGACTTATGAAAGACATTCTCTCTCTCATTTTGTTAACTTAGAC
AACCTGAGAGCTAATAACACTAAAGGTTCATTGCCTATTAATGTTATAGTTTTTGATGGTAAATCAAAAT
GTGAAGAATCATCTGCAAAATCAGCGTCTGTTTACTACAGTCAGCTTATGTGTCAACCTATACTGTTACT
AGATCAGGCATTAGTGTCTGATGTTGGTGATAGTGCGGAAGTTGCAGTTAAAATGTTTGATGCTTACGTT
AATACGTTTTCATCAACTTTTAACGTACCAATGGAAAAACTCAAAACACTAGTTGCAACTGCAGAAGCTG
AACTTGCAAAGAATGTGTCCTTAGACAATGTCTTATCTACTTTTATTTCAGCAGCTCGGCAAGGGTTTGT
TGATTCAGATGTAGAAACTAAAGATGTTGTTGAATGTCTTAAATTGTCACATCAATCTGACATAGAAGTT
ACTGGCGATAGTTGTAATAACTATATGCTCACCTATAACAAAGTTGAAAACATGACACCCCGTGACCTTG
GTGCTTGTATTGACTGTAGTGCGCGTCATATTAATGCGCAGGTAGCAAAAAGTCACAACATTGCTTTGAT
ATGGAACGTTAAAGATTTCATGTCATTGTCTGAACAACTACGAAAACAAATACGTAGTGCTGCTAAAAAG
AATAACTTACCTTTTAAGTTGACATGTGCAACTACTAGACAAGTTGTTAATGTTGTAACAACAAAGATAG
CACTTAAGGGTGGTAAAATTGTTAATAATTGGTTGAAGCAGTTAATTAAAGTTACACTTGTGTTCCTTTT
TGTTGCTGCTATTTTCTATTTAATAACACCTGTTCATGTCATGTCTAAACATACTGACTTTTCAAGTGAA
ATCATAGGATACAAGGCTATTGATGGTGGTGTCACTCGTGACATAGCATCTACAGATACTTGTTTTGCTA
ACAAACATGCTGATTTTGACACATGGTTTAGCCAGCGTGGTGGTAGTTATACTAATGACAAAGCTTGCCC
ATTGATTGCTGCAGTCATAACAAGAGAAGTGGGTTTTGTCGTGCCTGGTTTGCCTGGCACGATATTACGC
ACAACTAATGGTGACTTTTTGCATTTCTTACCTAGAGTTTTTAGTGCAGTTGGTAACATCTGTTACACAC
CATCAAAACTTATAGAGTACACTGACTTTGCAACATCAGCTTGTGTTTTGGCTGCTGAATGTACAATTTT
TAAAGATGCTTCTGGTAAGCCAGTACCATATTGTTATGATACCAATGTACTAGAAGGTTCTGTTGCTTAT
GAAAGTTTACGCCCTGACACACGTTATGTGCTCATGGATGGCTCTATTATTCAATTTCCTAACACCTACC
TTGAAGGTTCTGTTAGAGTGGTAACAACTTTTGATTCTGAGTACTGTAGGCACGGCACTTGTGAAAGATC
AGAAGCTGGTGTTTGTGTATCTACTAGTGGTAGATGGGTACTTAACAATGATTATTACAGATCTTTACCA
GGAGTTTTCTGTGGTGTAGATGCTGTAAATTTACTTACTAATATGTTTACACCACTAATTCAACCTATTG
GTGCTTTGGACATATCAGCATCTATAGTAGCTGGTGGTATTGTAGCTATCGTAGTAACATGCCTTGCCTA
CTATTTTATGAGGTTTAGAAGAGCTTTTGGTGAATACAGTCATGTAGTTGCCTTTAATACTTTACTATTC
CTTATGTCATTCACTGTACTCTGTTTAACACCAGTTTACTCATTCTTACCTGGTGTTTATTCTGTTATTT
ACTTGTACTTGACATTTTATCTTACTAATGATGTTTCTTTTTTAGCACATATTCAGTGGATGGTTATGTT
CACACCTTTAGTACCTTTCTGGATAACAATTGCTTATATCATTTGTATTTCCACAAAGCATTTCTATTGG
TTCTTTAGTAATTACCTAAAGAGACGTGTAGTCTTTAATGGTGTTTCCTTTAGTACTTTTGAAGAAGCTG
CGCTGTGCACCTTTTTGTTAAATAAAGAAATGTATCTAAAGTTGCGTAGTGATGTGCTATTACCTCTTAC
GCAATATAATAGATACTTAGCTCTTTATAATAAGTACAAGTATTTTAGTGGAGCAATGGATACAACTAGC
TACAGAGAAGCTGCTTGTTGTCATCTCGCAAAGGCTCTCAATGACTTCAGTAACTCAGGTTCTGATGTTC
TTTACCAACCACCACAAACCTCTATCACCTCAGCTGTTTTGCAGAGTGGTTTTAGAAAAATGGCATTCCC
ATCTGGTAAAGTTGAGGGTTGTATGGTACAAGTAACTTGTGGTACAACTACACTTAACGGTCTTTGGCTT
GATGACGTAGTTTACTGTCCAAGACATGTGATCTGCACCTCTGAAGACATGCTTAACCCTAATTATGAAG
ATTTACTCATTCGTAAGTCTAATCATAATTTCTTGGTACAGGCTGGTAATGTTCAACTCAGGGTTATTGG
ACATTCTATGCAAAATTGTGTACTTAAGCTTAAGGTTGATACAGCCAATCCTAAGACACCTAAGTATAAG
TTTGTTCGCATTCAACCAGGACAGACTTTTTCAGTGTTAGCTTGTTACAATGGTTCACCATCTGGTGTTT
ACCAATGTGCTATGAGGCCCAATTTCACTATTAAGGGTTCATTCCTTAATGGTTCATGTGGTAGTGTTGG
TTTTAACATAGATTATGACTGTGTCTCTTTTTGTTACATGCACCATATGGAATTACCAACTGGAGTTCAT
GCTGGCACAGACTTAGAAGGTAACTTTTATGGACCTTTTGTTGACAGGCAAACAGCACAAGCAGCTGGTA
CGGACACAACTATTACAGTTAATGTTTTAGCTTGGTTGTACGCTGCTGTTATAAATGGAGACAGGTGGTT
TCTCAATCGATTTACCACAACTCTTAATGACTTTAACCTTGTGGCTATGAAGTACAATTATGAACCTCTA
ACACAAGACCATGTTGACATACTAGGACCTCTTTCTGCTCAAACTGGAATTGCCGTTTTAGATATGTGTG
CTTCATTAAAAGAATTACTGCAAAATGGTATGAATGGACGTACCATATTGGGTAGTGCTTTATTAGAAGA
TGAATTTACACCTTTTGATGTTGTTAGACAATGCTCAGGTGTTACTTTCCAAAGTGCAGTGAAAAGAACA
ATCAAGGGTACACACCACTGGTTGTTACTCACAATTTTGACTTCACTTTTAGTTTTAGTCCAGAGTACTC
AATGGTCTTTGTTCTTTTTTTTGTATGAAAATGCCTTTTTACCTTTTGCTATGGGTATTATTGCTATGTC
TGCTTTTGCAATGATGTTTGTCAAACATAAGCATGCATTTCTCTGTTTGTTTTTGTTACCTTCTCTTGCC
ACTGTAGCTTATTTTAATATGGTCTATATGCCTGCTAGTTGGGTGATGCGTATTATGACATGGTTGGATA
TGGTTGATACTAGTTTGTCTGGTTTTAAGCTAAAAGACTGTGTTATGTATGCATCAGCTGTAGTGTTACT
AATCCTTATGACAGCAAGAACTGTGTATGATGATGGTGCTAGGAGAGTGTGGACACTTATGAATGTCTTG
ACACTCGTTTATAAAGTTTATTATGGTAATGCTTTAGATCAAGCCATTTCCATGTGGGCTCTTATAATCT
CTGTTACTTCTAACTACTCAGGTGTAGTTACAACTGTCATGTTTTTGGCCAGAGGTATTGTTTTTATGTG
TGTTGAGTATTGCCCTATTTTCTTCATAACTGGTAATACACTTCAGTGTATAATGCTAGTTTATTGTTTC
TTAGGCTATTTTTGTACTTGTTACTTTGGCCTCTTTTGTTTACTCAACCGCTACTTTAGACTGACTCTTG
GTGTTTATGATTACTTAGTTTCTACACAGGAGTTTAGATATATGAATTCACAGGGACTACTCCCACCCAA
GAATAGCATAGATGCCTTCAAACTCAACATTAAATTGTTGGGTGTTGGTGGCAAACCTTGTATCAAAGTA
GCCACTGTACAGTCTAAAATGTCAGATGTAAAGTGCACATCAGTAGTCTTACTCTCAGTTTTGCAACAAC
TCAGAGTAGAATCATCATCTAAATTGTGGGCTCAATGTGTCCAGTTACACAATGACATTCTCTTAGCTAA
AGATACTACTGAAGCCTTTGAAAAAATGGTTTCACTACTTTCTGTTTTGCTTTCCATGCAGGGTGCTGTA
GACATAAACAAGCTTTGTGAAGAAATGCTGGACAACAGGGCAACCTTACAAGCTATAGCCTCAGAGTTTA
GTTCCCTTCCATCATATGCAGCTTTTGCTACTGCTCAAGAAGCTTATGAGCAGGCTGTTGCTAATGGTGA
TTCTGAAGTTGTTCTTAAAAAGTTGAAGAAGTCTTTGAATGTGGCTAAATCTGAATTTGACCGTGATGCA
GCCATGCAACGTAAGTTGGAAAAGATGGCTGATCAAGCTATGACCCAAATGTATAAACAGGCTAGATCTG
AGGACAAGAGGGCAAAAGTTACTAGTGCTATGCAGACAATGCTTTTCACTATGCTTAGAAAGTTGGATAA
TGATGCACTCAACAACATTATCAACAATGCAAGAGATGGTTGTGTTCCCTTGAACATAATACCTCTTACA
ACAGCAGCCAAACTAATGGTTGTCATACCAGACTATAACACATATAAAAATACGTGTGATGGTACAACAT
TTACTTATGCATCAGCATTGTGGGAAATCCAACAGGTTGTAGATGCAGATAGTAAAATTGTTCAACTTAG
TGAAATTAGTATGGACAATTCACCTAATTTAGCATGGCCTCTTATTGTAACAGCTTTAAGGGCCAATTCT
GCTGTCAAATTACAGAATAATGAGCTTAGTCCTGTTGCACTACGACAGATGTCTTGTGCTGCCGGTACTA
CACAAACTGCTTGCACTGATGACAATGCGTTAGCTTACTACAACACAACAAAGGGAGGTAGGTTTGTACT
TGCACTGTTATCCGATTTACAGGATTTGAAATGGGCTAGATTCCCTAAGAGTGATGGAACTGGTACTATC
TATACAGAACTGGAACCACCTTGTAGGTTTGTTACAGACACACCTAAAGGTCCTAAAGTGAAGTATTTAT
ACTTTATTAAAGGATTAAACAACCTAAATAGAGGTATGGTACTTGGTAGTTTAGCTGCCACAGTACGTCT
ACAAGCTGGTAATGCAACAGAAGTGCCTGCCAATTCAACTGTATTATCTTTCTGTGCTTTTGCTGTAGAT
GCTGCTAAAGCTTACAAAGATTATCTAGCTAGTGGGGGACAACCAATCACTAATTGTGTTAAGATGTTGT
GTACACACACTGGTACTGGTCAGGCAATAACAGTTACACCGGAAGCCAATATGGATCAAGAATCCTTTGG
TGGTGCATCGTGTTGTCTGTACTGCCGTTGCCACATAGATCATCCAAATCCTAAAGGATTTTGTGACTTA
AAAGGTAAGTATGTACAAATACCTACAACTTGTGCTAATGACCCTGTGGGTTTTACACTTAAAAACACAG
TCTGTACCGTCTGCGGTATGTGGAAAGGTTATGGCTGTAGTTGTGATCAACTCCGCGAACCCATGCTTCA
GTCAGCTGATGCACAATCGTTTTTAAACGGGTTTGCGGTGTAAGTGCAGCCCGTCTTACACCGTGCGGCA
CAGGCACTAGTACTGATGTCGTATACAGGGCTTTTGACATCTACAATGATAAAGTAGCTGGTTTTGCTAA
ATTCCTAAAAACTAATTGTTGTCGCTTCCAAGAAAAGGACGAAGATGACAATTTAATTGATTCTTACTTT
GTAGTTAAGAGACACACTTTCTCTAACTACCAACATGAAGAAACAATTTATAATTTACTTAAGGATTGTC
CAGCTGTTGCTAAACATGACTTCTTTAAGTTTAGAATAGACGGTGACATGGTACCACATATATCACGTCA
ACGTCTTACTAAATACACAATGGCAGACCTCGTCTATGCTTTAAGGCATTTTGATGAAGGTAATTGTGAC
ACATTAAAAGAAATACTTGTCACATACAATTGTTGTGATGATGATTATTTCAATAAAAAGGACTGGTATG
ATTTTGTAGAAAACCCAGATATATTACGCGTATACGCCAACTTAGGTGAACGTGTACGCCAAGCTTTGTT
AAAAACAGTACAATTCTGTGATGCCATGCGAAATGCTGGTATTGTTGGTGTACTGACATTAGATAATCAA
GATCTCAATGGTAACTGGTATGATTTCGGTGATTTCATACAAACCACGCCAGGTAGTGGAGTTCCTGTTG
TAGATTCTTATTATTCATTGTTAATGCCTATATTAACCTTGACCAGGGCTTTAACTGCAGAGTCACATGT
TGACACTGACTTAACAAAGCCTTACATTAAGTGGGATTTGTTAAAATATGACTTCACGGAAGAGAGGTTA
AAACTCTTTGACCGTTATTTTAAATATTGGGATCAGACATACCACCCAAATTGTGTTAACTGTTTGGATG
ACAGATGCATTCTGCATTGTGCAAACTTTAATGTTTTATTCTCTACAGTGTTCCCACCTACAAGTTTTGG
ACCACTAGTGAGAAAAATATTTGTTGATGGTGTTCCATTTGTAGTTTCAACTGGATACCACTTCAGAGAG
CTAGGTGTTGTACATAATCAGGATGTAAACTTACATAGCTCTAGACTTAGTTTTAAGGAATTACTTGTGT
ATGCTGCTGACCCTGCTATGCACGCTGCTTCTGGTAATCTATTACTAGATAAACGCACTACGTGCTTTTC
AGTAGCTGCACTTACTAACAATGTTGCTTTTCAAACTGTCAAACCCGGTAATTTTAACAAAGACTTCTAT
GACTTTGCTGTGTCTAAGGGTTTCTTTAAGGAAGGAAGTTCTGTTGAATTAAAACACTTCTTCTTTGCTC
AGGATGGTAATGCTGCTATCAGCGATTATGACTACTATCGTTATAATCTACCAACAATGTGTGATATCAG
ACAACTACTATTTGTAGTTGAAGTTGTTGATAAGTACTTTGATTGTTACGATGGTGGCTGTATTAATGCT
AACCAAGTCATCGTCAACAACCTAGACAAATCAGCTGGTTTTCCATTTAATAAATGGGGTAAGGCTAGAC
TTTATTATGATTCAATGAGTTATGAGGATCAAGATGCACTTTTCGCATATACAAAACGTAATGTCATCCC
TACTATAACTCAAATGAATCTTAAGTATGCCATTAGTGCAAAGAATAGAGCTCGCACCGTAGCTGGTGTC
TCTATCTGTAGTACTATGACCAATAGACAGTTTCATCAAAAATTATTGAAATCAATAGCCGCCACTAGAG
GAGCTACTGTAGTAATTGGAACAAGCAAATTCTATGGTGGTTGGCACAACATGTTAAAAACTGTTTATAG
TGATGTAGAAAACCCTCACCTTATGGGTTGGGATTATCCTAAATGTGATAGAGCCATGCCTAACATGCTT
AGAATTATGGCCTCACTTGTTCTTGCTCGCAAACATACAACGTGTTGTAGCTTGTCACACCGTTTCTATA
GATTAGCTAATGAGTGTGCTCAAGTATTGAGTGAAATGGTCATGTGTGGCGGTTCACTATATGTTAAACC
AGGTGGAACCTCATCAGGAGATGCCACAACTGCTTATGCTAATAGTGTTTTTAACATTTGTCAAGCTGTC
ACGGCCAATGTTAATGCACTTTTATCTACTGATGGTAACAAAATTGCCGATAAGTATGTCCGCAATTTAC
AACACAGACTTTATGAGTGTCTCTATAGAAATAGAGATGTTGACACAGACTTTGTGAATGAGTTTTACGC
ATATTTGCGTAAACATTTCTCAATGATGATACTCTCTGACGATGCTGTTGTGTGTTTCAATAGCACTTAT
GCATCTCAAGGTCTAGTGGCTAGCATAAAGAACTTTAAGTCAGTTCTTTATTATCAAAACAATGTTTTTA
TGTCTGAAGCAAAATGTTGGACTGAGACTGACCTTACTAAAGGACCTCATGAATTTTGCTCTCAACATAC
AATGCTAGTTAAACAGGGTGATGATTATGTGTACCTTCCTTACCCAGATCCATCAAGAATCCTAGGGGCC
GGCTGTTTTGTAGATGATATCGTAAAAACAGATGGTACACTTATGATTGAACGGTTCGTGTCTTTAGCTA
TAGATGCTTACCCACTTACTAAACATCCTAATCAGGAGTATGCTGATGTCTTTCATTTGTACTTACAATA
CATAAGAAAGCTACATGATGAGTTAACAGGACACATGTTAGACATGTATTCTGTTATGCTTACTAATGAT
AACACTTCAAGGTATTGGGAACCTGAGTTTTATGAGGCTATGTACACACCGCATACAGTCTTACAGGCTG
TTGGGGCTTGTGTTCTTTGCAATTCACAGACTTCATTAAGATGTGGTGCTTGCATACGTAGACCATTCTT
ATGTTGTAAATGCTGTTACGACCATGTCATATCAACATCACATAAATTAGTCTTGTCTGTTAATCCGTAT
GTTTGCAATGCTCCAGGTTGTGATGTCACAGATGTGACTCAACTTTACTTAGGAGGTATGAGCTATTATT
GTAAATCACATAAACCACCCATTAGTTTTCCATTGTGTGCTAATGGACAAGTTTTTGGTTTATATAAAAA
TACATGTGTTGGTAGCGATAATGTTACTGACTTTAATGCAATTGCAACATGTGACTGGACAAATGCTGGT
GATTACATTTTAGCTAACACCTGTACTGAAAGACTCAAGCTTTTTGCAGCAGAAACGCTCAAAGCTACTG
AGGAGACATTTAAACTGTCTTATGGTATTGCTACTGTACGTGAAGTGCTGTCTGACAGAGAATTACATCT
TTCATGGGAAGTTGGTAAACCTAGACCACCACTTAACCGAAATTATGTCTTTACTGGTTATCGTGTAACT
AAAAACAGTAAAGTACAAATAGGAGAGTACACCTTTGAAAAAGGTGACTATGGTGATGCTGTTGTTTACC
GAGGTACAACAACTTACAAATTAAATGTTGGTGATTATTTTGTGCTGACATCACATACAGTAATGCCATT
AAGTGCACCTACACTAGTGCCACAAGAGCACTATGTTAGAATTACTGGCTTATACCCAACACTCAATATC
TCAGATGAGTTTTCTAGCAATGTTGCAAATTATCAAAAGGTTGGTATGCAAAAGTATTCTACACTCCAGG
GACCACCTGGTACTGGTAAGAGTCATTTTGCTATTGGCCTAGCTCTCTACTACCCTTCTGCTCGCATAGT
GTATACAGCTTGCTCTCATGCCGCTGTTGATGCACTATGTGAGAAGGCATTAAAATATTTGCCTATAGAT
AAATGTAGTAGAATTATACCTGCACGTGCTCGTGTAGAGTGTTTTGATAAATTCAAAGTGAATTCAACAT
TAGAACAGTATGTCTTTTGTACTGTAAATGCATTGCCTGAGACGACAGCAGATATAGTTGTCTTTGATGA
AATTTCAATGGCCACAAATTATGATTTGAGTGTTGTCAATGCCAGATTACGTGCTAAGCACTATGTGTAC
ATTGGCGACCCTGCTCAATTACCTGCACCACGCACATTGCTAACTAAGGGCACACTAGAACCAGAATATT
TCAATTCAGTGTGTAGACTTATGAAAACTATAGGTCCAGACATGTTCCTCGGAACTTGTCGGCGTTGTCC
TGCTGAAATTGTTGACACTGTGAGTGCTTTGGTTTATGATAATAAGCTTAAAGCACATAAAGACAAATCA
GCTCAATGCTTTAAAATGTTTTATAAGGGTGTTATCACGCATGATGTTTCATCTGCAATTAACAGGCCAC
AAATAGGCGTGGTAAGAGAATTCCTTACACGTAACCCTGCTTGGAGAAAAGCTGTCTTTATTTCACCTTA
TAATTCACAGAATGCTGTAGCCTCAAAGATTTTGGGACTACCAACTCAAACTGTTGATTCATCACAGGGC
TCAGAATATGACTATGTCATATTCACTCAAACCACTGAAACAGCTCACTCTTGTAATGTAAACAGATTTA
ATGTTGCTATTACCAGAGCAAAAGTAGGCATACTTTGCATAATGTCTGATAGAGACCTTTATGACAAGTT
GCAATTTACAAGTCTTGAAATTCCACGTAGGAATGTGGCAACTTTACAAGCTGAAAATGTAACAGGACTC
TTTAAAGATTGTAGTAAGGTAATCACTGGGTTACATCCTACACAGGCACCTACACACCTCAGTGTTGACA
CTAAATTCAAAACTGAAGGTTTATGTGTTGACATACCTGGCATACCTAAGGACATGACCTATAGAAGACT
CATCTCTATGATGGGTTTTAAAATGAATTATCAAGTTAATGGTTACCCTAACATGTTTATCACCCGCGAA
GAAGCTATAAGACATGTACGTGCATGGATTGGCTTCGATGTCGAGGGGTGTCATGCTACTAGAGAAGCTG
TTGGTACCAATTTACCTTTACAGCTAGGTTTTTCTACAGGTGTTAACCTAGTTGCTGTACCTACAGGTTA
TGTTGATACACCTAATAATACAGATTTTTCCAGAGTTAGTGCTAAACCACCGCCTGGAGATCAATTTAAA
CACCTCATACCACTTATGTACAAAGGACTTCCTTGGAATGTAGTGCGTATAAAGATTGTACAAATGTTAA
GTGACACACTTAAAAATCTCTCTGACAGAGTCGTATTTGTCTTATGGGCACATGGCTTTGAGTTGACATC
TATGAAGTATTTTGTGAAAATAGGACCTGAGCGCACCTGTTGTCTATGTGATAGACGTGCCACATGCTTT
TCCACTGCTTCAGACACTTATGCCTGTTGGCATCATTCTATTGGATTTGATTACGTCTATAATCCGTTTA
TGATTGATGTTCAACAATGGGGTTTTACAGGTAACCTACAAAGCAACCATGATCTGTATTGTCAAGTCCA
TGGTAATGCACATGTAGCTAGTTGTGATGCAATCATGACTAGGTGTCTAGCTGTCCACGAGTGCTTTGTT
AAGCGTGTTGACTGGACTATTGAATATCCTATAATTGGTGATGAACTGAAGATTAATGCGGCTTGTAGAA
AGGTTCAACACATGGTTGTTAAAGCTGCATTATTAGCAGACAAATTCCCAGTTCTTCACGACATTGGTAA
CCCTAAAGCTATTAAGTGTGTACCTCAAGCTGATGTAGAATGGAAGTTCTATGATGCACAGCCTTGTAGT
GACAAAGCTTATAAAATAGAAGAATTATTCTATTCTTATGCCACACATTCTGACAAATTCACAGATGGTG
TATGCCTATTTTGGAATTGCAATGTCGATAGATATCCTGCTAATTCCATTGTTTGTAGATTTGACACTAG
AGTGCTATCTAACCTTAACTTGCCTGGTTGTGATGGTGGCAGTTTGTATGTAAATAAACATGCATTCCAC
ACACCAGCTTTTGATAAAAGTGCTTTTGTTAATTTAAAACAATTACCATTTTTCTATTACTCTGACAGTC
CATGTGAGTCTCATGGAAAACAAGTAGTGTCAGATATAGATTATGTACCACTAAAGTCTGCTACGTGTAT
AACACGTTGCAATTTAGGTGGTGCTGTCTGTAGACATCATGCTAATGAGTACAGATTGTATCTCGATGCT
TATAACATGATGATCTCAGCTGGCTTTAGCTTGTGGGTTTACAAACAATTTGATACTTATAACCTCTGGA
ACACTTTTACAAGACTTCAGAGTTTAGAAAATGTGGCTTTTAATGTTGTAAATAAGGGACACTTTGATGG
ACAACAGGGTGAAGTACCAGTTTCTATCATTAATAACACTGTTTACACAAAAGTTGATGGTGTTGATGTA
GAATTGTTTGAAAATAAAACAACATTACCTGTTAATGTAGCATTTGAGCTTTGGGCTAAGCGCAACATTA
AACCAGTACCAGAGGTGAAAATACTCAATAATTTGGGTGTGGACATTGCTGCTAATACTGTGATCTGGGA
CTACAAAAGAGATGCTCCAGCACATATATCTACTATTGGTGTTTGTTCTATGACTGACATAGCCAAGAAA
CCAACTGAAACGATTTGTGCACCACTCACTGTCTTTTTTGATGGTAGAGTTGATGGTCAAGTAGACTTAT
TTAGAAATGCCCGTAATGGTGTTCTTATTACAGAAGGTAGTGTTAAAGGTTTACAACCATCTGTAGGTCC
CAAACAAGCTAGTCTTAATGGAGTCACATTAATTGGAGAAGCCGTAAAAACACAGTTCAATTATTATAAG
AAAGTTGATGGTGTTGTCCAACAATTACCTGAAACTTACTTTACTCAGAGTAGAAATTTACAAGAATTTA
AACCCAGGAGTCAAATGGAAATTGATTTCTTAGAATTAGCTATGGATGAATTCATTGAACGGTATAAATT
AGAAGGCTATGCCTTCGAACATATCGTTTATGGAGATTTTAGTCATAGTCAGTTAGGTGGTTTACATCTA
CTGATTGGACTAGCTAAACGTTTTAAGGAATCACCTTTTGAATTAGAAGATTTTATTCCTATGGACAGTA
CAGTTAAAAACTATTTCATAACAGATGCGCAAACAGGTTCATCTAAGTGTGTGTGTTCTGTTATTGATTT
ATTACTTGATGATTTTGTTGAAATAATAAAATCCCAAGATTTATCTGTAGTTTCTAAGGTTGTCAAAGTG
ACTATTGACTATACAGAAATTTCATTTATGCTTTGGTGTAAAGATGGCCATGTAGAAACATTTTACCCAA
AATTACAATCTAGTCAAGCGTGGCAACCGGGTGTTGCTATGCCTAATCTTTACAAAATGCAAAGAATGCT
ATTAGAAAAGTGTGACCTTCAAAATTATGGTGATAGTGCAACATTACCTAAAGGCATAATGATGAATGTC
GCAAAATATACTCAACTGTGTCAATATTTAAACACATTAACATTAGCTGTACCCTATAATATGAGAGTTA
TACATTTTGGTGCTGGTTCTGATAAAGGAGTTGCACCAGGTACAGCTGTTTTAAGACAGTGGTTGCCTAC
GGGTACGCTGCTTGTCGATTCAGATCTTAATGACTTTGTCTCTGATGCAGATTCAACTTTGATTGGTGAT
TGTGCAACTGTACATACAGCTAATAAATGGGATCTCATTATTAGTGATATGTACGACCCTAAGACTAAAA
ATGTTACAAAAGAAAATGACTCTAAAGAGGGTTTTTTCACTTACATTTGTGGGTTTATACAACAAAAGCT
AGCTCTTGGAGGTTCCGTGGCTATAAAGATAACAGAACATTCTTGGAATGCTGATCTTTATAAGCTCATG
GGACACTTCGCATGGTGGACAGCCTTTGTTACTAATGTGAATGCGTCATCATCTGAAGCATTTTTAATTG
GATGTAATTATCTTGGCAAACCACGCGAACAAATAGATGGTTATGTCATGCATGCAAATTACATATTTTG
GAGGAATACAAATCCAATTCAGTTGTCTTCCTATTCTTTATTTGACATGAGTAAATTTCCCCTTAAATTA
AGGGGTACTGCTGTTATGTCTTTAAAAGAAGGTCAAATCAATGATATGATTTTATCTCTTCTTAGTAAAG
GTAGACTTATAATTAGAGAAAACAACAGAGTTGTTATTTCTAGTGATGTTCTTGTTAACAACTAAACGAA
CAATGTTTGTTTTTCTTGTTTTATTGCCACTAGTCTCTAGTCAGTGTGTTAATCTTACAACCAGAACTCA
ATTACCCCCTGCATACACTAATTCTTTCACACGTGGTGTTTATTACCCTGACAAAGTTTTCAGATCCTCA
GTTTTACATTCAACTCAGGACTTGTTCTTACCTTTCTTTTCCAATGTTACTTGGTTCCATGCTATACATG
TCTCTGGGACCAATGGTACTAAGAGGTTTGATAACCCTGTCCTACCATTTAATGATGGTGTTTATTTTGC
TTCCACTGAGAAGTCTAACATAATAAGAGGCTGGATTTTTGGTACTACTTTAGATTCGAAGACCCAGTCC
CTACTTATTGTTAATAACGCTACTAATGTTGTTATTAAAGTCTGTGAATTTCAATTTTGTAATGATCCAT
TTTTGGGTGTTTATTACCACAAAAACAACAAAAGTTGGATGGAAAGTGAGTTCAGAGTTTATTCTAGTGC
GAATAATTGCACTTTTGAATATGTCTCTCAGCCTTTTCTTATGGACCTTGAAGGAAAACAGGGTAATTTC
AAAAATCTTAGGGAATTTGTGTTTAAGAATATTGATGGTTATTTTAAAATATATTCTAAGCACACGCCTA
TTAATTTAGTGCGTGATCTCCCTCAGGGTTTTTCGGCTTTAGAACCATTGGTAGATTTGCCAATAGGTAT
TAACATCACTAGGTTTCAAACTTTACTTGCTTTACATAGAAGTTATTTGACTCCTGGTGATTCTTCTTCA
GGTTGGACAGCTGGTGCTGCAGCTTATTATGTGGGTTATCTTCAACCTAGGACTTTTCTATTAAAATATA
ATGAAAATGGAACCATTACAGATGCTGTAGACTGTGCACTTGACCCTCTCTCAGAAACAAAGTGTACGTT
GAAATCCTTCACTGTAGAAAAAGGAATCTATCAAACTTCTAACTTTAGAGTCCAACCAACAGAATCTATT
GTTAGATTTCCTAATATTACAAACTTGTGCCCTTTTGGTGAAGTTTTTAACGCCACCAGATTTGCATCTG
TTTATGCTTGGAACAGGAAGAGAATCAGCAACTGTGTTGCTGATTATTCTGTCCTATATAATTCCGCATC
ATTTTCCACTTTTAAGTGTTATGGAGTGTCTCCTACTAAATTAAATGATCTCTGCTTTACTAATGTCTAT
GCAGATTCATTTGTAATTAGAGGTGATGAAGTCAGACAAATCGCTCCAGGGCAAACTGGAAAGATTGCTG
ATTATAATTATAAATTACCAGATGATTTTACAGGCTGCGTTATAGCTTGGAATTCTAACAATCTTGATTC
TAAGGTTGGTGGTAATTATAATTACCTGTATAGATTGTTTAGGAAGTCTAATCTCAAACCTTTTGAGAGA
GATATTTCAACTGAAATCTATCAGGCCGGTAGCACACCTTGTAATGGTGTTGAAGGTTTTAATTGTTACT
TTCCTTTACAATCATATGGTTTCCAACCCACTAATGGTGTTGGTTACCAACCATACAGAGTAGTAGTACT
TTCTTTTGAACTTCTACATGCACCAGCAACTGTTTGTGGACCTAAAAAGTCTACTAATTTGGTTAAAAAC
AAATGTGTCAATTTCAACTTCAATGGTTTAACAGGCACAGGTGTTCTTACTGAGTCTAACAAAAAGTTTC
TGCCTTTCCAACAATTTGGCAGAGACATTGCTGACACTACTGATGCTGTCCGTGATCCACAGACACTTGA
GATTCTTGACATTACACCATGTTCTTTTGGTGGTGTCAGTGTTATAACACCAGGAACAAATACTTCTAAC
CAGGTTGCTGTTCTTTATCAGGATGTTAACTGCACAGAAGTCCCTGTTGCTATTCATGCAGATCAACTTA
CTCCTACTTGGCGTGTTTATTCTACAGGTTCTAATGTTTTTCAAACACGTGCAGGCTGTTTAATAGGGGC
TGAACATGTCAACAACTCATATGAGTGTGACATACCCATTGGTGCAGGTATATGCGCTAGTTATCAGACT
CAGACTAATTCTCCTCGGCGGGCACGTAGTGTAGCTAGTCAATCCATCATTGCCTACACTATGTCACTTG
GTGCAGAAAATTCAGTTGCTTACTCTAATAACTCTATTGCCATACCCACAAATTTTACTATTAGTGTTAC
CACAGAAATTCTACCAGTGTCTATGACCAAGACATCAGTAGATTGTACAATGTACATTTGTGGTGATTCA
ACTGAATGCAGCAATCTTTTGTTGCAATATGGCAGTTTTTGTACACAATTAAACCGTGCTTTAACTGGAA
TAGCTGTTGAACAAGACAAAAACACCCAAGAAGTTTTTGCACAAGTCAAACAAATTTACAAAACACCACC
AATTAAAGATTTTGGTGGTTTTAATTTTTCACAAATATTACCAGATCCATCAAAACCAAGCAAGAGGTCA
TTTATTGAAGATCTACTTTTCAACAAAGTGACACTTGCAGATGCTGGCTTCATCAAACAATATGGTGATT
GCCTTGGTGATATTGCTGCTAGAGACCTCATTTGTGCACAAAAGTTTAACGGCCTTACTGTTTTGCCACC
TTTGCTCACAGATGAAATGATTGCTCAATACACTTCTGCACTGTTAGCGGGTACAATCACTTCTGGTTGG
ACCTTTGGTGCAGGTGCTGCATTACAAATACCATTTGCTATGCAAATGGCTTATAGGTTTAATGGTATTG
GAGTTACACAGAATGTTCTCTATGAGAACCAAAAATTGATTGCCAACCAATTTAATAGTGCTATTGGCAA.
AATTCAAGACTCACTTTCTTCCACAGCAAGTGCACTTGGAAAACTTCAAGATGTGGTCAACCAAAATGCA
CAAGCTTTAAACACGCTTGTTAAACAACTTAGCTCCAATTTTGGTGCAATTTCAAGTGTTTTAAATGATA
TCCTTTCACGTCTTGACAAAGTTGAGGCTGAAGTGCAAATTGATAGGTTGATCACAGGCAGACTTCAAAG
TTTGCAGACATATGTGACTCAACAATTAATTAGAGCTGCAGAAATCAGAGCTTCTGCTAATCTTGCTGCT
ACTAAAATGTCAGAGTGTGTACTTGGACAATCAAAAAGAGTTGATTTTTGTGGAAAGGGCTATCATCTTA
TGTCCTTCCCTCAGTCAGCACCTCATGGTGTAGTCTTCTTGCATGTGACTTATGTCCCTGCACAAGAAAA
GAACTTCACAACTGCTCCTGCCATTTGTCATGATGGAAAAGCACACTTTCCTCGTGAAGGTGTCTTTGTT
TCAAATGGCACACACTGGTTTGTAACACAAAGGAATTTTTATGAACCACAAATCATTACTACAGACAACA
CATTTGTGTCTGGTAACTGTGATGTTGTAATAGGAATTGTCAACAACACAGTTTATGATCCTTTGCAACC
TGAATTAGACTCATTCAAGGAGGAGTTAGATAAATATTTTAAGAATCATACATCACCAGATGTTGATTTA
GGTGACATCTCTGGCATTAATGCTTCAGTTGTAAACATTCAAAAAGAAATTGACCGCCTCAATGAGGTTG
CCAAGAATTTAAATGAATCTCTCATCGATCTCCAAGAACTTGGAAAGTATGAGCAGTATATAAAATGGCC
ATGGTACATTTGGCTAGGTTTTATAGCTGGCTTGATTGCCATAGTAATGGTGACAATTATGCTTTGCTGT
ATGACCAGTTGCTGTAGTTGTCTCAAGGGCTGTTGTTCTTGTGGATCCTGCTGCAAATTTGATGAAGACG
ACTCTGAGCCAGTGCTCAAAGGAGTCAAATTACATTACACATAAACGAACTTATGGATTTGTTTATGAGA
ATCTTCACAATTGGAACTGTAACTTTGAAGCAAGGTGAAATCAAGGATGCTACTCCTTCAGATTTTGTTC
GCGCTACTGCAACGATACCGATACAAGCCTCACTCCCTTTCGGATGGCTTATTGTTGGCGTTGCACTTCT
TGCTGTTTTTCAGAGCGCTTCCAAAATCATAACCCTCAAAAAGAGATGGCAACTAGCACTCTCCAAGGGT
GTTCACTTTGTTTGCAACTTGCTGTTGTTGTTTGTAACAGTTTACTCACACCTTTTGCTCGTTGCTGCTG
GCCTTGAAGCCCCTTTTCTCTATCTTTATGCTTTAGTCTACTTCTTGCAGAGTATAAACTTTGTAAGAAT
AATAATGAGGCTTTGGCTTTGCTGGAAATGCCGTTCCAAAAACCCATTACTTTATGATGCCAACTATTTT
CTTTGCTGGCATACTAATTGTTACGACTATTGTATACCTTACAATAGTGTAACTTCTTCAATTGTCATTA
CTTCAGGTGATGGCACAACAAGTCCTATTTCTGAACATGACTACCAGATTGGTGGTTATACTGAAAAATG
GGAATCTGGAGTAAAAGACTGTGTTGTATTACACAGTTACTTCACTTCAGACTATTACCAGCTGTACTCA
ACTCAATTGAGTACAGACACTGGTGTTGAACATGTTACCTTCTTCATCTACAATAAAATTGTTGATGAGC
CTGAAGAACATGTCCAAATTCACACAATCGACGGTTCATCCGGAGTTGTTAATCCAGTAATGGAACCAAT
TTATGATGAACCGACGACGACTACTAGCGTGCCTTTGTAAGCACAAGCTGATGAGTACGAACTTATGTAC
TCATTCGTTTCGGAAGAGACAGGTACGTTAATAGTTAATAGCGTACTTCTTTTTCTTGCTTTCGTGGTAT
TCTTGCTAGTTACACTAGCCATCCTTACTGCGCTTCGATTGTGTGCGTACTGCTGCAATATTGTTAACGT
GAGTCTTGTAAAACCTTCTTTTTACGTTTACTCTCGTGTTAAAAATCTGAATTCTTCTAGAGTTCCTGAT
CTTCTGGTCTAAACGAACTAAATATTATATTAGTTTTTCTGTTTGGAACTTTAATTTTAGCCATGGCAGA
TTCCAACGGTACTATTACCGTTGAAGAGCTTAAAAAGCTCCTTGAACAATGGAACCTAGTAATAGGTTTC
CTATTCCTTACATGGATTTGTCTTCTACAATTTGCCTATGCCAACAGGAATAGGTTTTTGTATATAATTA
AGTTAATTTTCCTCTGGCTGTTATGGCCAGTAACTTTAGCTTGTTTTGTGCTTGCTGCTGTTTACAGAAT
AAATTGGATCACCGGTGGAATTGCTATCGCAATGGCTTGTCTTGTAGGCTTGATGTGGCTCAGCTACTTC
ATTGCTTCTTTCAGACTGTTTGCGCGTACGCGTTCCATGTGGTCATTCAATCCAGAAACTAACATTCTTC
TCAACGTGCCACTCCATGGCACTATTCTGACCAGACCGCTTCTAGAAAGTGAACTCGTAATCGGAGCTGT
GATCCTTCGTGGACATCTTCGTATTGCTGGACACCATCTAGGACGCTGTGACATCAAGGACCTGCCTAAA
GAAATCACTGTTGCTACATCACGAACGCTTTCTTATTACAAATTGGGAGCTTCGCAGCGTGTAGCAGGTG
ACTCAGGTTTTGCTGCATACAGTCGCTACAGGATTGGCAACTATAAATTAAACACAGACCATTCCAGTAG
CAGTGACAATATTGCTTTGCTTGTACAGTAAGTGACAACAGATGTTTCATCTCGTTGACTTTCAGGTTAC
TATAGCAGAGATATTACTAATTATTATGAGGACTTTTAAAGTTTCCATTTGGAATCTTGATTACATCATA
AACCTCATAATTAAAAATTTATCTAAGTCACTAACTGAGAATAAATATTCTCAATTAGATGAAGAGCAAC
CAATGGAGATTGATTAAACGAACATGAAAATTATTCTTTTCTTGGCACTGATAACACTCGCTACTTGTGA
GCTTTATCACTACCAAGAGTGTGTTAGAGGTACAACAGTACTTTTAAAAGAACCTTGCTCTTCTGGAACA
TACGAGGGCAATTCACCATTTCATCCTCTAGCTGATAACAAATTTGCACTGACTTGCTTTAGCACTCAAT
TTGCTTTTGCTTGTCCTGACGGCGTAAAACACGTCTATCAGTTACGTGCCAGATCAGTTTCACCTAAACT
GTTCATCAGACAAGAGGAAGTTCAAGAACTTTACTCTCCAATTTTTCTTATTGTTGCGGCAATAGTGTTT
ATAACACTTTGCTTCACACTCAAAAGAAAGACAGAATGATTGAACTTTCATTAATTGACTTCTATTTGTG
CTTTTTAGCCTTTCTGCTATTCCTTGTTTTAATTATGCTTATTATCTTTTGGTTCTCACTTGAACTGCAA
GATCATAATGAAACTTGTCACGCCTAAACGAACATGAAATTTCTTGTTTTCTTAGGAATCATCACAACTG
TAGCTGCATTTCACCAAGAATGTAGTTTACAGTCATGTACTCAACATCAACCATATGTAGTTGATGACCC
GTGTCCTATTCACTTCTATTCTAAATGGTATATTAGAGTAGGAGCTAGAAAATCAGCACCTTTAATTGAA
TTGTGCGTGGATGAGGCTGGTTCTAAATCACCCATTCAGTACATCGATATCGGTAATTATACAGTTTCCT
GTTTACCTTTTACAATTAATTGCCAGGAACCTAAATTGGGTAGTCTTGTAGTGCGTTGTTCGTTCTATGA
AGACTTTTTAGAGTATCATGACGTTCGTGTTGTTTTAGATTTCATCTAAACGAACAAACTAAAATGTCTG
ATAATGGACCCCAAAATCAGCGAAATGCACCCCGCATTACGTTTGGTGGACCCTCAGATTCAACTGGCAG
TAACCAGAATGGAGAACGCAGTGGGGCGCGATCAAAACAACGTCGGCCCCAAGGTTTACCCAATAATACT
GCGTCTTGGTTCACCGCTCTCACTCAACATGGCAAGGAAGACCTTAAATTCCCTCGAGGACAAGGCGTTC
CAATTAACACCAATAGCAGTCCAGATGACCAAATTGGCTACTACCGAAGAGCTACCAGACGAATTCGTGG
TGGTGACGGTAAAATGAAAGATCTCAGTCCAAGATGGTATTTCTACTACCTAGGAACTGGGCCAGAAGCT
GGACTTCCCTATGGTGCTAACAAAGACGGCATCATATGGGTTGCAACTGAGGGAGCCTTGAATACACCAA
AAGATCACATTGGCACCCGCAATCCTGCTAACAATGCTGCAATCGTGCTACAACTTCCTCAAGGAACAAC
ATTGCCAAAAGGCTTCTACGCAGAAGGGAGCAGAGGCGGCAGTCAAGCCTCTTCTCGTTCCTCATCACGT
AGTCGCAACAGTTCAAGAAATTCAACTCCAGGCAGCAGTAGGGGAACTTCTCCTGCTAGAATGGCTGGCA
ATGGCGGTGATGCTGCTCTTGCTTTGCTGCTGCTTGACAGATTGAACCAGCTTGAGAGCAAAATGTCTGG
TAAAGGCCAACAACAACAAGGCCAAACTGTCACTAAGAAATCTGCTGCTGAGGCTTCTAAGAAGCCTCGG
CAAAAACGTACTGCCACTAAAGCATACAATGTAACACAAGCTTTCGGCAGACGTGGTCCAGAACAAACCC
AAGGAAATTTTGGGGACCAGGAACTAATCAGACAAGGAACTGATTACAAACATTGGCCGCAAATTGCACA
ATTTGCCCCCAGCGCTTCAGCGTTCTTCGGAATGTCGCGCATTGGCATGGAAGTCACACCTTCGGGAACG
TGGTTGACCTACACAGGTGCCATCAAATTGGATGACAAAGATCCAAATTTCAAAGATCAAGTCATTTTGC
TGAATAAGCATATTGACGCATACAAAACATTCCCACCAACAGAGCCTAAAAAGGACAAAAAGAAGAAGGC
TGATGAAACTCAAGCCTTACCGCAGAGACAGAAGAAACAGCAAACTGTGACTCTTCTTCCTGCTGCAGAT
TTGGATGATTTCTCCAAACAATTGCAACAATCCATGAGCAGTGCTGACTCAACTCAGGCCTAAACTCATG
CAGACCACACAAGGCAGATGGGCTATATAAACGTTTTCGCTTTTCCGTTTACGATATATAGTCTACTCTT
GTGCAGAATGAATTCTCGTAACTACATAGCACAAGTAGATGTAGTTAACTTTAATCTCACATAGCAATCT
TTAATCAGTGTGTAACATTAGGGAGGACTTGAAAGAGCCACCACATTTTCACCGAGGCCACGCGGAGTAC
GATCGAGTGTACAGTGAACAATGCTAGGGAGAGCTGCCTATATGGAAGAGCCCTAATGTGTAAAATTAAT
TTTAGTAGTGCTATCCCCATGTGATTTTAATAGCTTCTTAGGAGAATGACAAAAAAAAAAAAAAAAAAAA
AAAAAAAAAAAAA

Coronavirus VLPs can be generated by co-expression of the structural proteins of coronaviruses (S, M, N and E), in which there can be one or more mutations in one or more the structural proteins, as well as packaging signal (see Syed et al, Science 3374, 1626-1632 (2010; which is incorporated herein in its entirety). Exemplary plasmids of the disclosure include plasmids that allow expression of viral proteins, such as SARS-CoV-2 structural proteins. In particular embodiments, the plasmids include a promoter and a terminator sequence.

### Variant Analysis

A library of expression plasmids coding for mutant N, S, E, M proteins or a combination thereof, are transduced in cells, such as 293T. Cells can be expanded and transfected with plasmids expressing the remaining SARS-CoV-2 proteins, E, M, N or S. For example, if the 293T cells were transduced with mutant S proteins, then after expansion they would be transfected with one or more plasmids expressing M, N or E proteins. Therefore, after transduction and transfection, each cell expresses all four SARS-CoV-2 proteins (all four proteins can be mutants or there can be a combination of mutant and wild type proteins). At some point (either before, after or along with transduction of plasmids coding for the mutant protein, the cells can be transduced to carry a packaging signal, such as PS9 (packaging signal 9).

An exemplary protocol for transduction/transfection includes preparation of plasmid transduction/transfection mixture including appropriate media, plasmids, and a transfection agent (e.g., lipofectamine). The transduction/transfection mixture is incubated with cells to be transfected (e.g., 293T cells) for a period time (e.g., overnight) under appropriate conditions (e.g., 37° C. and 5% CO₂).

Supernatants from the cells carrying at least the five components discussed above (S, M, N, E and PS9; initial cells) can be used to infect other cells (secondary cells), such as cells that overexpress the ACE2 and/or TMPRSS2, including 293T ACE2 TMPRSS2 cells (such as those that be purchased from Creative Biogene; studies have shown that SARS-CoV-2 uses the SARS-CoV receptor ACE2 for entry and the serine protease TMPRSS2 for S protein priming).

Deep mutational scanning, which combines functional selection with high throughout sequencing (for example, 10⁴ to 10⁵ variants of a protein can be constructed, and selection of function can be imposed) can measure how mutations to SARS-CoV-2, such as mutations in N, M, E and S proteins, affect viral growth in cell culture, viral neutralization by antibodies, and viral neutralization by polyclonal human sera. This analysis can improve forecasting viral evolution and guiding of the development of future vaccines and antivirals. Additionally, the variant libraries generated disclosed herein can be used to map the epitopes of SARS-CoV-2 binding antibodies; to inform antibody drug development by characterizing mutations in target viral proteins that allow development of viral resistance to antibodies; and/or to assess the ability of different viral entry proteins to evade antibody neutralization, overcome drug inhibition, and/or infect new species.

To assess neutralization escape, the VLP pool (see FIG. 1), will be preincubated with serum from vaccinated subjects (e.g., humans) before infection of cells. RNA can then be extracted from the VLP pool (prior to infection) and infected cells and PacBio sequenced (or other massively parallel long-read sequencing methods) to determine enrichment of each variant (e.g., Spike variant, such as enrichment of Spike variant sequences in infected cells compared to original viral inoculum). For extremely large library sizes, the library will be barcoded and the barcodes will be linked to variants using PacBio sequencing. The sequencing readout following an experiment will be using short-read Illumina-based technologies to determine the abundance of barcodes in the VLP pool (prior to infection) and in infected cells.

The following Example illustrates some of the materials, methods, and experiments that were used or performed in the development of the invention.

### Example

### Mutational Constraints on Particle Production and Infectivity Impact SARS-CoV-2 Spike Protein Evolution/Deep Mutational Scan of SARS-CoV-2 Virus-Like Particles Reveals Constraints of Spike Mutations on Viral Particle Production and Infectivity.

### Introduction

A major challenge in curbing the COVID-19 pandemic has been the continued emergence of SARS-CoV-2 variants with enhanced infectivity and neutralization escape (Ref: Nat Med. 2022 March; 28(3):481-485. And BA 2.86 cell 2 papers 2024). Updated booster vaccines have been rolled out but were already outdated by the time of distribution (MMWR Morb Mortal Wkly Rep 2022; 71:1526-1530; N Engl J Med 2023; 388:565-567; N Engl J Med 2022; 386:1910-1921; N Engl J Med 2022; 387:1279-1291). A barrier to rapid and accurate booster vaccine deployment is the lag time between variant characterization and roll-out of the booster. Proactive characterization of viral variants and a solid knowledge of what mutations enhance and impair infection are needed to provide sufficient lead time to develop relevant and timely booster vaccines.

Several lines of research have successfully initiated this process, utilizing Spike receptor binding domain (RBD) yeast surface display (Starr et al. Cell. Volume 182, Issue 5, P1295-1310.E20, 2020) and Spike pseudotyped lentiviral particles to assess the contribution of Spike mutations to ACE2 binding and cellular entry (Dadonaite et al., Cell vol. 186, issue 6, 1263-1278, 2023), respectively. They have provided invaluable data to study SARS-CoV-2 Spike protein evolutionary trajectories (Dadonaite, et al., 2023. bioRxiv, Full-spike deep mutational scanning helps predict the evolutionary success of SARS-CoV-2 clades; doi: https://doi.org/10.1101/2023.11.13.566961), epistasis in emerging variants (Id.), and efficacy and escape mutants of monoclonal antibodies (Dodonaite et al. vol. 186, issue 6, P1263-1278.E20. 2023). However, an important knowledge gap remains as structural proteins such as Spike not only fulfill entry requirements but might also be critical for the process of viral particle production. Current methods cannot provide accurate information for this process as they either do not form viral particles (yeast display) or rely on assembly of non-SARS-CoV-2 particles (pseudotyped particles). We have previously described a SARS-CoV-2 virus-like particle (VLP) system (Syed Science) that faithfully recapitulates SARS-CoV-2 assembly (Syed Science, Johnson PLOS Pathogens) as well as provides authentic information about variant entry (Syed PNAS), and antibody neutralization (Syed PNAS and Syed Cell). Herein, this method was adapted to a high throughput format and a deep mutational scan was conducted of the Spike receptor-binding domain (RBD) which mediates ACE2-receptor binding, viral entry and antibody neutralization. Provided herein are previously unrecognized mutational constraints within the RBD on virion production and infectivity and these restraints are mapped to defects in Spike processing.

### Materials and Methods

### Molecular Cloning

All plasmids utilized for standard VLP generation including pSpike, pMIRESE, pN R203M, and pLuc PS9 were described previously. All spike mutants for validation experiments were generated on the pSpike plasmid using HiFi DNA assembly. Plasmids PSPax2 and pMD2.G were described previously and obtained via Addgene. To generate the Spike PS9 lentiviral vector for the deep mutational scan, we started with pLVX-EF1-IRES-Puro construct and inserted the Spike and PS9 sequences using HiFi DNA assembly. Further, the EF1a intron was removed using HiFi DNA assembly to reduce the lentiviral packaged RNA and improve lentiviral titers for transduction. Lastly, to ensure that no background Spike PS9 construct remains after cloning in the deep mutational scan library, two PaqCI restriction sites were inserted flanking the RBD sequence and that are out of frame of the Spike coding sequence. Therefore, if the vector self-ligates, the Spike sequence would contain multiple stop codons and not express in transduced cells.

To generate a deep mutational scan library of the Spike RBD, Twist Biosciences' Site Saturation Library spanning the entire 201 aa of the RBD as used. The library was ordered as pooled double stranded DNA fragments (150 ng) and was cloned into 330 ng of PaqCI digested and gel-purified Spike PS9 lentiviral vector using HiFi DNA assembly in a 20 uL reaction. After 1 hour at 50° C., 0.4 uL 5M NaCl, 0.2 uL Glycoblue, and 20 uL of isopropanol were added. The solution was mixed well and incubated at room temperature for 15 minutes. The DNA was pelleted by centrifugation at 20 kg for 15 min at 4° C. The pellet was washed twice with 80% EtOH and air dried for 5 minutes. The pellet was resuspended in 5 uL TE and 2 uL was utilized for electroporating 50 uL of EnduraMax electrocompetent cells in two cuvettes per manufacturer's recommendation. The bacteria were outgrown for 1 hour at 37° C. in 2 mL recovery medium each. The bacteria were plated on two large square agar plates and also diluted 1:10000 and plated on a 10 cm agar plate. The bacteria were grown at 30° C. for 20 hours and had an estimated recovery of 1.2×10⁷ CFU, while control plates (no library insert during assembly) had an estimated recovery of 1×10⁴. The colonies were scraped and maxiprepred and utilized for subsequent experiments (Machery Nagel).

### Cells

293T and BHK-21 cells were obtained from ATCC and grown in DMEM supplemented with 10% FBS, 1× non-essential amino acids, 1× Glutamax, 10 mM HEPES, and 1× pen-strep at 37° C. in a humidified environment and 5% CO₂. 293T cells stably expressing ACE2 TMPRSS2 were generated as described previously and were grown as 293T cells with the addition of 10 ng/ml blasticidin and 250 ug Hygromycin.

### VLP Production and Spike RBD PCR

To generate VLPs from 293T cells, a previously described procedure was followed. Briefly, 293T cells were seeded to be at 50% confluency the day of transfection. In a 24-well plate, the cells were transfected with 6.25 ng pSpike, 165 ng pMIRESE, 333 ng pN, and 500 ng pLuc PS9 using Xtremegene 9 transfection reagent at a 1:3 ratio per manufacturer's recommendation. The media was changed 12-16 hours post-transfection. The cells were grown for an additional 48 hours prior to supernatant collection and 0.45 um filtration. For luciferase readout, the supernatant was mixed with trypsinized 293T ACE2 TMPRSS2 cells and plated in white 96-well plate overnight. The cells were washed gently with PBS and lysed with Passive Lysis Buffer and incubated at room temperature for 15 minutes. 50 uL of Firefly luciferase reagent was added to each well and read immediately on a Tecan plate reader. For PCR analysis, the supernatant was mixed with 293T ACE2 TMPRSS2 cells and plated in a 24-well plate. 12 hours after plating, the cells were washed gently with PBS and 200 uL of RNAstat60 was added. RNA was extracted using Zymo prep kit according to the manufacturer's recommendation. Reverse transcription was done using SuperScript IV kit using the primer-specific RT protocol using primer 5′ AGCTGGCGCAGATTCCAG 3′ (SEQ ID NO: 6). The cDNA was utilized directly for Taq PCR according to the manufacturer's recommendations or qPCR analysis. Primers were utilized to amplify the Spike RBD using forward primer 5′ cctccaatttecgegtgcagc 3′ (SEQ ID NO: 7) and reverse primer 5′ gttccggtcaggccgttgaaattg 3′ (SEQ ID NO: 8).

### Deep Mutational Scan Experiments

To generate VSV-G pseudotyped lentiviral pool bearing the DMS library in the Spike PS9 vector, 293T cells 80-90% confluent were transfected in two duplicate 15-cm dish with 9.86 ug pMD2.G, 15.08 ug PSPax2, and 19.72 ug of the Spike PS9 library construct using Xtremegene 9 transfection reagent (1:3 ratio). The media was changed 12-16 hours post-transfection. 48 hr after transfection, the supernatant was collected, and 0.45 um filtered. The goal was to transduce at least 6.67×10⁶ cells at 0.3 MOI to obtain at least 100× coverage of the theoretical library size. To achieve this, 1×10⁷ 293T cells were mixed with 0, 0.3, 1, 3, 10 mL of the lentiviral pool supernatant and the cells were plated in 15-cm dishes with 10 ug/mL polybrene After 24 hours, the media was replaced with fresh medium containing 1.5 ug/mL puromycin. It was previously determined that this is the minimum concentration necessary to kill all cells within 2-3 days. This is necessary to avoid high concentrations of puromycin which may select for high expressing mutants and reduce library diversity. After 4-6 days of selection, the 1 mL lentiviral treated cells were selected to continue to the next step as the cells were only 20-30% viable compared to no puromycin control, which is the desired MOI. The cells were expanded for another 2 days and then plated in 10-cm plate. The cells were transfected with 13.32 ug pMIRESE and 26.68 ug pN R203M using Xtremegene 9 transfection reagent (1.3 ratio). The media was changed 12-16 hours post transfection. After 48 hours, the supernatant (10 mL) was collected and 0.45 um filtered and the cells were washed with PBS and collected in RNAstat60 and frozen at −80° C. 0.5 mL of the supernatant was mixed with 1 mL RNAstat60 and stored at −80 C. 3 mL of the supernatant was mixed with 7.5×10⁵ 293T ACE2 TMPRSS2 cells and plated in 6-well plate. An additional control containing no VLPs was plated as a negative control. After 10-12 hours, the cells were washed with PBS and collected in RNAstat60 and stored in −80° C. The entire procedure above was repeated the subsequent week to obtain a total of two independent biological replicates containing two technical replicates each. The RNA was extracted from all samples and reverse transcription was done as described in the VLP production and Spike RBD PCR section. The PCR was conducted using Roche HiFi PCR kit according to the manufacturer's instructions. The amplicons were purified with XP beads and utilized for PacBio SMRTbell library preparation according to the manufacturer's instructions. The amplicons from producer cells, VLPs, and infected cells were barcoded and pooled for each technical replicate for a total of four pools. Each pool was sequenced on a full Sequel Ile flow cell.

### Computational Analysis of DMS Results

Demultiplexed PacBio reads were trimmed to eliminate primer sequences and filtered to only the appropriate RBD nucleotide sequence size using Geneious Prime. The filtered reads were aligned to the wild-type RBD sequence using Geneious Prime. The mapped reads were then utilized for variant calling using Enrich2 software as previously described.

Enrich2 provides a tsv with mutants in one column and read counts in the next column. Each replicate was on a different flow cell in the sequencer. The replicates were integrated.

### VLP Validation Experiments

293T cells were seeded at 7×10⁶ cells per 15-cm dish and transfected the next day (50% confluent) with 6.64 ug pMIRESE, 13.32 ug pN R203M, 20 ug pLuc PS9, and 0.4 ug pSpike variant plasmids using Xtremegene 9 transfection reagent (1:3 ratio). The media was changed 12-16 hr post-transfection. After 48 hours, the supernatant was collected and 0.45 um filtered. The cells were washed with PBS and collected in RIPA lysis buffer and stored at −20° C. The supernatant was spun in an SW32Ti over 3 mL 20% sucrose cushion at 24 krpm for 2 hours at 4° C. The tube was inverted and the pellet was dissolved in 100 uL PBS at 4° C. for 15 minutes. For infectivity, 1 ul of VLP was diluted in 175 ul of complete media, then 50 ul was used to infect 1×10⁴ HEK293T-ACE2-TMPRSS2 cells in triplicates. After 12-24 hr, the cells were washed with PBS and lysed in Passive Lysis Buffer for 15 min at room temperature. 50 uL of luciferase assay reagent was added to each well and luminescence was measured using a Tecan plate reader. The luciferase values were first normalized to the WT Spike luciferase signal to account for variability across different experiments. In a second normalization step, each values was then normalized to the amount of Spike on the particle measured by Western blot analysis (FIG. 4B) and plotted in FIG. 4D as “VLP infectivity.” For mutants resulting in bald particles (C379S, L387P, L387Q, R454H, R457C) or almost bald particles (A352P, D467G), the luciferase values were not normalized to avoid artifacts as they were at background levels and the amount of Spike on the particle was also at background levels.

For Western blot analysis, 12 ul of VLP was added to 4×NuPAGE dye (with B-mercaptoethanol), boiled at 95° C. for 5 min, ran on 4-20% Mini-PROTEAN® TGX™ Precast Protein Gels (BioRad, Cat: 4561096) and transferred to a 0.2 um Nitrocellulose membrane. The membrane was blocked in 10% NFDM and stained with primary antibody: anti-N(Sino Biologicals 40143-MM05, 1:1000 dilution), anti-S (abcam ab272504, 1:500), anti-GAPDH (Cell Signaling $174S, 1:1000), anti-p24 (Sigma SAB3500946, 1:2000) for O/N at 4° C. Blots were rinsed with TBS-T three times for 5 minutes each and stained with secondary HRP antibody (Bethyl A90-516P (mouse), A120-201P (rabbit) 1:5000). Imaged using a chemiluminescence kit (Roche 12015200001, Thermo Scientific™ 34096). Image was captured using ChemiDoc™ Imaging System (Biorad 12003153). Densitometry was done using ImageJ software.

### Deglycosylation Experiments

For Spike deglycosylation, 40 ug of cell lysate was treated with PNGaseF (NEB, P0709S) and EndoH (NEB, P0702S) according to the manufacturer's protocol. Briefly, the samples were prepared in glycoprotein denaturation buffer and denatured at 100° C. for 10 mins. Then, 2 ul of PGF or 5 ul of EndoH was added with their respective buffer and suggested reagents. The samples were incubated at 37° C. for 2 hours and analyzed by SDS-PAGE and Western blot.

### Immunoprecipitation Experiments

HEK293T cells were seeded with 5×10⁵ cells/well. Spike expression vector was co-transfected with Strep-tagged M or N with Xtremegene 9 transfection reagent (1:3 ratio). At the 48 hr time point, cell lysate was collected and lysed in IP buffer (50 mM Tris-HCl, 150 mM NaCl, 1 mM EDTA, 1% NP40, supplemented with Halt protease inhibitor cocktail). For pulldown, 0.5 mg of lysate was incubated overnight with 30 ul of Strep-Tactin Sepharose resin (IBA Life Science, 2-1201-002), rotating at 4° C. Bound protein was washed six times with IP buffer and eluted with 1× Strep-Tactin elution buffer (IBA Life Sciences, 2-1000-025). Eluted samples were analyzed by western blot. For strep-tagged protein detection, strep antibody (IBA Life Sciences, IBA-2-1507-001) was used.

### Replicon Validation Experiments

The replicon experiments were conducted as previously described. Briefly, BHK21 cells were seeded at 5×10⁴ per well in 24-well plates. The next day, the cells (30-50% confluent) were transfected with 1 ug of pBAC WA1 delS replicon construct, 0.5 ug pN R203M, and 0.5 ug pSpike variant plasmids using Xtremegene 9 transfection reagent (1:3 ratio). The media was changed 12-16 hours post-transfection. After 48 hours, the supernatant was collected and filtered using 96-well Supor 0.45 um filter. 150 uL of the supernatant was mixed with 4×10⁴ 293T ACE2 TMPRSS2 cells and plated in 96-well plates in triplicate. After 12-16 hours, the cells were washed with PBS and 100 uL of complete growth medium added. After 48 hours, 50 uL of the supernatant was transferred to a white 96-well plate and 50 uL of nanoLuc reagent was added to each well. The luciferase signal was read on a Tecan plate reader.

### Results

### Generation of a High-Throughput VLP Platform

To conduct a deep mutational scan that requires interrogation of thousands of mutations simultaneously, a method to package RNA encoding Spike into VLPs was first developed. A previously identified the PS9 sequence in SARS-CoV-2 (nt 20,080-21,171) was identified as the sequence necessary and sufficient for RNA packaging (Syed Science). Herein is the generation of Spike RNA fusion with PS9 to package Spike coding sequences into SARS-CoV-2 VLPs. Stable “producer” cell lines were generated expressing either Spike or Spike-PS9 that assembled particles after transient introduction of the other structural proteins N, M, and E (Supplementary FIGS. S1A and S1B) The previously described luciferase-PS9 reporter RNA was added for rapid assessment of particle production and entry into “receiver” cells (Supplementary FIGS. S1A and S1B). Notably, particles generated in stable Spike-PS9 cell lines carry the same Spike sequence as cargo as the Spike protein decorating their surface, allowing one to record the abundance of a certain Spike sequence in a receiver cell as surrogate of how well the same Spike protein performed in viral fitness. The Spike PS9 cells generated ˜5-fold lower VLPs, which was interpreted as a sign of competition in packaging between the Spike-PS9 and luciferase-PS9 transcripts. It was confirmed by RT-PCR that VLPs generated in Spike-PS9 cells successfully delivered the Spike-PS9 transcript to cells in a receptor-dependent manner as the signal was only detected in cells expressing ACE2 and TMPRSS2 (FIG. 1A-C). Supernatant that was heat inactivated and/or RNAse treated did not yield a positive signal in receiver cells indicating successful incorporation into particles (FIG. 1A-C). In addition, a reverse transcription (RT) step was necessary to detect the Spike PS9 transcript by PCR indicating that the particles delivered RNA and not contaminating genomic DNA from producer cells (FIG. 1A-C).

VLP production was optimized in stable Spike-PS9 cells by using a matrix of different concentrations of N and M and E expression vectors (FIGS. 1D and 1E) as well as testing different time points (Supplementary FIG. S2). Lastly, VLPs were pelleted by ultracentrifugation and it was confirmed that the Spike-PS9 transcript was delivered in a particle-dependent manner (FIG. 1F). Collectively, these experiments produced an optimized VLP platform that can efficiently package and deliver Spike transcripts into authentic SARS-CoV-2 particles that are decorated with the encoded Spike proteins.

Deep Mutational Scanning of the Spike RBD with VLPs

The advantage of the VLP platform is that it can systematically interrogate mutations in any region of Spike or other structural proteins of SARS-CoV-2. The Spike RBD was focused on first as deep mutational scans have been performed using alternative techniques and because it is a focus of virus evolution due to its role in ACE2 binding and its antigenic properties. To perform a deep mutational scan, a site-saturation library of the Spike RBD was constructed and cloned into the Spike-PS9 construct, VSV-G pseudotyped the library, and transduced the library into 293T cells to generate VLP producer cells (FIG. 2A). After puromycin selection, the polyclonal cell population was transiently transfected with expression vectors of N and M and E. The supernatant was collected after 72 hours and used to infect 293T cells stably expressing ACE2 TMPRSS2. RNA from the receiver cells was extracted, reverse transcribed, PCR amplified, and PacBio sequenced (FIG. 2A). In addition, RNA was collected from the producer cells as well as the supernatant to control for appropriate RNA expression and packaging, respectively. A total of four independent biological experiments were conducted for a total of four data sets of each condition: Producer cells, VLPs in supernatant, and receiver cells. A mean of ˜650K PacBio reads was obtained after filtering and mapping to the RBD sequence (Supplementary FIG. S3). The producer cells contained on average 3814 (99.87%) unique single point variants of the total 3819 desired variants (FIG. 2B). Synonymous mutations were utilized as wild-type sequences in the library. There were an average 1816 unique variants with 2+ mutations and only an average of 18 variants with stop codons (FIG. 2B), both with relatively rare sequence abundance in producer cells. The producer cell counts were normally distributed with an average of ˜100 counts per variant (FIG. 2C and Supplementary FIG. S4). However, the average counts per variant were higher in VLPs than in receiver cells and there was a decrease in the number of unique variants compared with producer cells, indicating a dramatic loss in library diversity at the VLP production and to a lesser extent infection steps (FIG. 2C).

To quantify the impact of mutations on VLP production, the enrichment of each mutation in the VLPs pool relative to the producer cells was calculated as a log ratio (FIG. 3A) of VLP counts to producer cell counts (Supplementary FIG. 4). Notably, the majority of mutations resulted in lower VLP production compared with the wild-type sequence (FIG. 3C). In addition, numerous mutations were not detected in the VLP pool suggesting significant reductions in particle production or technical issues with sequencing the RNA in the supernatant. Given that most sequences are normally distributed in producer cells (FIG. 2C), it was hypothesized that lower counts in producer cells may result in lower counts in VLPs, thus lower assembly scores. To test this hypothesis, the distribution of producer cell counts was analyzed and compared to those with high VLP production (>500 counts in VLPs), with those with low VLP production (<10 counts in VLPs). Variants with high VIP production tended to have higher counts in producer cells (FIG. 3E), but variants with low VLP production did not show such correlation and did not deviate in their distribution of producer cell counts from all single variants together (FIG. 3C) These data indicate that RBD mutations that lower VLP production might affect viral assembly through changes at the Spike protein level.

The enrichment of each mutation in receiver cells was calculated as the log ratio (FIG. 3B) of receiver cell counts to producer cell counts (Suppl FIG. S4). Surprisingly, the unique number of variants detected in receiver cells was much greater than those detected in VLPs. This suggests that the reduced library diversity observed in the VLP pool is likely due to technical issues with sequencing RNA in the supernatant. In addition, the majority of variants had a negative impact on VLP entry (FIG. 3D) and in general VLP entry enrichment values between 0 and −5 appear to be much more variable than those that are lower than-5 or higher than 0 (FIG. 3F). Interestingly, correlation between the VLP production and VLP entry datasets did not reveal any obvious trends (FIG. 3G). Correlations between the VLP entry data and the RBD expression and ACE2 binding data in the yeast surface display system were conducted and it was found that the data correlate slightly better with ACE2 binding than RBD expression (FIG. 3H). That is, Spike RBD mutants with low ACE2 binding tend to consistently have lower VLP entry, but mutants with higher ACE2 binding tend to have variable VLP entry (FIG. 3H). These data indicate that the VLP assay likely incorporates other processes mediating entry including, but not exclusively, ACE2 binding. Similar correlations were conducted with recent pseudotype virus (PTV) entry data and low correlations were found suggesting potentially different phenotypes of mutants between the two different systems. Of note, the PTV system utilizes a C-terminally truncated Spike to enable robust assembly onto lentiviral particles, while the VLP system utilizes full length Spike. Differences in assembly may explain the poor correlation between the two datasets. Collectively, the DMS VLP platform is functional and versatile, revealing significant mutational constraints of the Spike RBD on particle assembly and infectivity.

### Spike RBD Controls Viral Assembly

The significant impact of Spike RBD mutations on particle production was unexpected and further experiments were focused on its validation. A subset of mutants were selected as a cross section of phenotypes observed for VLP production and entry and individual VLP studies were performed (FIG. 4A). None of the selected mutations significantly impacted the abundance of the N protein in purified VLPs indicating that in all cases, particles were successfully produced (FIG. 4B). However, many mutations resulted in low (A352P, V341S, S438Y, Y396H, F464C, G485V, D467G, E406K, F400L, G339S) to no (C379S, L387P, L387Q, R454H, R457C) Spike protein detected on purified VLPs although all were similarly expressed in transfected cells (FIG. 4B) This indicates that certain mutations induce “bald particle” that Jack Spike at their surface that are detectable by sequencing but are not infectious. Of note, bald particles have been described for other coronaviruses (doi: 10. 1128/JVI.01131-10).

When mutations inducing “bald particles” were excluded, the DMS assembly measurement and Western blot Spike measurements in individual VLPs correlated well (Spearman's r=0.55). Infectivity of the selected mutants in VLP assays (FIG. 4A) and replicons were tested, where a Spike-deleted near-full-length viral sequence is complemented with an individual Spike sequence to produce single-round infectious particles (Suppl FIG. S5A). Bald particles with Spike RBD mutations C379S, L387P, L387Q, R454H, and R457C were not infectious in these assays as expected. Results from VLP assay correlated well with results from replicon assays (r=0.60) validating VLPs as physiological testing platform (Suppl FIG. S4B). Collectively, these findings identify a novel role of Spike RBD mutations in Spike assembly onto viral particles.

Additionally, it was sought to determine the abundance of low-assembly mutants (Supplementary FIG. 6A) in natural sequences on the GISAID database (Supplementary FIG. 6B) as well as in a fitness model developed by Bloom and Neher (Supplementary FIG. 6C) (https://doi.org/10.1101/2023.01.30.526314). It was found that low-assembly mutants were typically absent or found at low abundance (<10 sequences) on the GISAID database and typically had low or undetermined fitness scores (Supplementary FIG. 6A-C). This potentially suggests that the observed deleterious effects for these mutants prevent them from being acquired in natural sequences. Interestingly, some of the high-assembly mutants were sometimes absent from natural sequences, such as V362S (Supplementary FIG. 6A-C). A possible explanation could be that this mutation has a different phenotype when combined with other RBD mutations in natural sequences due to epistasis (https://doi.org/10.1126/science.abo7896).

### The RBD Plays a Role in Spike Processing

The Spike protein is heavily glycosylated, and its proper transit through the ER and Golgi organelles is necessary for its maturation, cleavage, and assembly onto particles. A first clue that Spike glycosylation was affected came from the banding pattern of Spike proteins in western blotting (FIG. 4B). Spike usually shows two bands, the S and S2 bands representing the products of furin cleavage (https://doi.org/10.1371/journal.ppat.1009246). Low-assembly mutants characteristically had no “smearing” of the S band and absence of S2 band, which is observed with the wild-type Spike and high-assembly mutants, potentially indicative of Spike glycosylation (FIG. 4B). It was hypothesized that the low-assembly mutations prevent Spike glycosylation, leading to a loss of mature Spike in particles. Indeed, digestion of cellular extracts with PNGase F (an amidase that cleaves almost all N-linked oligosaccharides), but not Endo H (a glycosidase that cleaves high mannose and some hybrid oligosaccharides), demonstrated that the smearing is due to glycosylation as after digestion all samples showed similar S banding (FIGS. 5A and 5B). Interestingly, the S2 band was very faint in low-assembly mutants, suggesting that proper glycosylation and maturation of Spike is necessary for efficient cleavage. This was not observed in a recent study where mutations of individual asparagines in the RBD that serve as glycosylation sites left Spike banding patterns unchanged but significantly impacted Spike assembly onto the virus particle (https://doi.org/10.1128/mbio.01672-23). Collectively, these data suggest that a global defect in Spike glycosylation or processing might be induced due to RBD mutations.

To determine further whether Spike is fully passing through the ER Golgi or is misfolded and localized in the ER, immunofluorescence studies were conducted of cells producing VLPs with wild-type as well as high-assembly mutant V362S and low-assembly mutants L387Q and F400L. It was found that the wild-type and V362S Spike proteins have punctate pattern and co-localize with the nucleocapsid protein, potentially at VLP assembly sites (FIG. 5C). Interestingly, the low-assembly mutants L387Q and F400L had a diffuse pattern that co-localized with an ER marker GRP78 suggesting a potentially misfolded protein that is not transiting the ER/Golgi system (FIG. 5C). This would consequently prevent the glycosylation and cleavage of these Spike proteins and prevent them from reaching VLP assembly sites. Taken together, these data demonstrate that the low-assembly Spike proteins appear to have a global misfolding event that results in significantly reduced processing.

Coronavirus assembly has been studied extensively (https://doi.org/10.1128/mBio.02371-21) but how Spike is assembled onto the particle and how glycosylation regulates this process is unclear. In SARS-CoV-2, the N protein's interaction with the RNA is thought to play a nucleating role with protein:protein interactions supporting the process (https://doi.org/10.1101%2F2023.11.22.568361). The E and M proteins have been shown to reduce Spike processing and increase its retention in the ER (https://doi.org/10.1074/jbc.RA120.016175). Of note, all experiments in FIG. 4 were performed in the presence of all structural proteins. To determine whether coexpression of the M or N proteins alone affected the glycosylation pattern of Spike, Western blotting was performed in co-transfected cells (Supplementary FIG. 7A). Spike mutants were selected that assemble similarly (V362S and N501T), lowly (F400L and S438Y), or not at all (L387Q) relative to the wild-type Spike. Only the characteristic Spike banding pattern in cells coexpressing N or the eGFP control were observed, but not M protein (Supplementary FIG. 7B). This indicates that the M protein plays a regulatory role in the processing of Spike and underscores the importance of characterizing Spike mutations in the presence of the full panel of SARS-CoV-2 structural proteins.

### Discussion

Provided herein is a novel VLP-based platform that importantly extends the current repertoire of variant-interrogating technologies by adding authentic particle production and assembly capabilities. Using this platform in a Spike RBD DMS novel mutations were found that restrain viral fitness at the level of assembly. In particular, amino acid sites C379, L387, R454, and R457 were sensitive to any amino acid mutations.

The results newly link the RBD to regulation of Spike glycosylation and loading onto the particle as bald particles were identified as a common cause of assembly deficit. Given the dramatic decrease in Spike glycosylation for individual point mutants, it appears that low-assembly mutations modulate the majority of Spike glycosylation. This suggests that these mutants are not modulating glycosylation of a single aspargine but possibly have a protein-wide effect which prevents processing. A possible mechanism is that these mutations hinder Spike maturation through the ER/Golgi thereby preventing glycosylation and assembly of the Spike protein onto particles.

Interestingly, it was found that co-expression of the Nucleocapsid and Spike proteins alone results in similar processing of the Spike protein to that in cells expressing all viral structural proteins. This is in line with a report showing that E and M cause retention of Spike in the ER/Golgi and prevent its maturation, while the Nucleocapsid protein does not. In addition, it was found that RBD mutants which do not assemble well lack interaction with the Nucleocapsid protein but not the Membrane protein. These data point to a potential role of the Nucleocapsid protein in regulating proper Spike glycosylation and processing and mediating its assembly onto the viral particle.

Labs around the world have contributed invaluable sequencing data of SARS-CoV-2 infections around the world. As of December 2023, over 16 million sequences have been deposited into public databases. This has served as a tool to study the evolutionary trajectories of SARS-CoV-2. Interestingly, it was found that mutations associated with bald particles in the instant assay were very rare in the public data bases (Suppl FIGS. S5A and S5B). Moreover, a fitness model that was developed by Bloom et al. based on over 6 million SARS-CoV-2 sequences showed either incalculable or significantly low fitness for mutations associated with bald particles in the assay (Suppl FIGS. S5A and S5C).

When comparing the instant findings to others, it was found that all the residues leading to poor assembly showed significant reduction in expression and ACE2 binding in a yeast display RBD system. Some mutations (L387P and E406K) were also previously assayed in a lentiviral-based Spike assay and showed reduced cellular entry effects. This points to the possibility that assembly and entry requirements in Spike are partially overlapping with glycosylation affecting both. Interestingly, no mutations that had both higher assembly and infectivity scores as compared to the wild-type sequence were found. This indicates that residues involved in viral assembly and entry may differ in their requirements for optimal execution of both functions. Existing sequences may represent a fragile equilibrium between both properties with mutations being mostly disruptive, shifting optimal performance towards one function at the cost of the other. The fact that individual mutations may function differently in the context of a mutated sequence and that epistatic effects heavily regulate functional outcomes represents a known limitation to every DMS, and combination studies are required to solve higher-order mutational restrains of the Spike protein or any other structural protein.

### BIBLIOGRAPHY

1. X. Xie et al., Cell Host Microbe 27, 841-848.e3 (2020).
2. S. Torii et al., Cell Rep. 35, 109014 (2021).
3. C. Ye et al., mBio 11, e02168-20 (2020).
4. X. Xie et al., Nat. Protoc. 16, 1761-1784 (2021).
5. S. J. Rihn et al., PLOS Biol. 19, e3001091 (2021).
6. J. A. Plante et al., Nature 592, 116-121 (2021).
7. K. H. D. Crawford et al., Viruses 12, 513 (2020).
8. A. A. Latif, Lineage comparison. outbreak.info (accessed 22 Jul. 2021); https://outbreak.info/compare-lineages.
9. W. Zeng et al., Biochem. Biophys. Res. Commun. 527, 618-623 (2020).
10. J. Cubuk et al., Nat. Commun. 12, 1936 (2021).
11. T. M. Perdikari et al., EMBO J. 39, e106478 (2020).
12. C. B. Plescia et al., J. Biol. Chem. 296, 100103 (2021).
13. H. Swann et al., Sci. Rep. 10, 21877 (2020).
14. J. Lu et al., Cell Res. 30, 936-939 (2020).
15. Y. L. Siu et al., J. Virol. 82, 11318-11330 (2008).
16. P.-K. Hsieh et al., J. Virol. 79, 13848-13855 (2005).
17. S. Dent, B. W. Neuman, Coronaviruses 1282, 99-108 (2015).
18. X. Lu et al., Immunology 122, 496-502 (2007).
19. L. Kuo, P. S. Masters, J. Virol. 87, 5182-5192 (2013).
20. K. Woo, M. Joo, K. Narayanan, K. H. Kim, S. Makino, J. Virol. 71, 824-827 (1997).
21. J. A. Fosmire, K. Hwang, S. Makino, J. Virol. 66, 3522-3530 (1992).
22. A. Kanakan et al., Pathogens 9, 912 (2020).
23. T. Giroglou et al., J. Virol. 78, 9007-9015 (2004).
24. M. Ujike, C. Huang, K. Shirato, S. Makino, F. Taguchi, J. Gen. Virol. 97, 1853-1864 (2016).
25. X. Deng et al., Cell 184, 3426-3437.e8 (2021).
26. A. Kuzmina et al., Cell Host Microbe 29, 522-528.e2 (2021).
27. Y. Liu et al., Nature 10.1038/s41586-021-04245-0 (2021).
28. C. Motozono et al., Cell Host Microbe 29, 1124-1136.e11 (2021).
29. R. Kalkeri et al., Microorganisms 9, 1744 (2021).
30. Y. Weisblum et al., eLife 9, e61312 (2020).
31. W. T. Harvey et al., Nat. Rev. Microbiol. 19, 409-424 (2021).
32. X. Ju et al., PLOS Pathog. 17, e1009439 (2021).
33. C. R. Carlson et al., Mol. Cell 80, 1092-1103.e4 (2020).
34. S. Lu et al., Nat. Commun. 12, 502 (2021).
35. B. Li et al., medRxiv 2021.07.07.21260122 [Preprint] (2021); doi: 10.1101/2021.07.07.21260122.
Syed et al., Science 374, 1626-1632 (2021)
Dadonaite et al., bioRxiv 2022.10.13.512056 [Preprint] Oct. 13, 2022.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed is:

1. A method to identify mutations in one or more SARS-CoV-2 structural proteins that affect infectivity comprising:
a) obtaining a library of mutants for at least one SARS-CoV-2 structural protein,
b) generating a virus like particle (VLP) pool by transducing plasmids expressing SARS-CoV-2 structural proteins E, M, N and S and a cis-acting RNA sequence that triggers packaging in initial cells, wherein at least one structural protein in each VLP has at least one of the mutants from the library of a),
c) infecting secondary cells by contacting said secondary cells with supernatant from the initial cell culture of b), and
d) sequencing the viral sequences in the initial and secondary cells to identify mutations in the said at least one structural protein that affect the infectivity,
wherein the viral sequences in the secondary cells of c) correlate with infectivity and the viral sequences in the initial cells of b) but not c) correlate with decreased infectivity.

2. A method to identify mutations in one or more SARS-CoV-2 structural proteins that affect sensitivity of said virus to a selection pressure comprising:
a) obtaining a library of mutants for at least one SARS-CoV-2 structural protein,
b) generating a virus like particle (VLP) pool by transducing plasmids expressing SARS-CoV-2 structural proteins E, M, N and S and a cis-acting RNA sequence that triggers packaging into initial cells and culturing, wherein at least one structural protein in each VLP has at least one of the mutants from the library of a),
c) infecting secondary cells by contacting said secondary cells with supernatant from the initial cell culture of b), wherein the initial cells are exposed to a selection pressure prior to infecting said secondary cells, and
d) sequencing the viral sequences in the secondary cells to identify mutations in the said at least one structural protein that affect the sensitivity of said virus to said selection pressure.

3. A method to map escape sites in an epitope of a structural SARS-CoV-2 protein from neutralizing antibodies comprising:
a) obtaining a library of mutants for at least one SARS-CoV-2 structural protein,
b) generating a virus like particles (VLP) pool by transducing plasmids expressing SARS-CoV-2 structural proteins E, M, N and S and a cis-acting RNA sequence that triggers packaging into initial cells and culturing, wherein at least one structural protein in each VLP has at least one of the mutants from the library of a),
c) infecting secondary cells by contacting said secondary cells with supernatant from the initial cell culture of b), wherein the initial cells are exposed to neutralizing antibodies prior to infecting said secondary cells, and
d) sequencing the viral sequences in the secondary cells to identify mutations in the said at least one structural protein that escaped the neutralizing antibodies.

4. The method of claim 1, wherein the mutant structural protein is S.

5. The method of claim 1, wherein the mutant structural protein is N.

6. The method of claim 1, wherein the mutant structural protein is M.

7. The method of claim 1, wherein the mutant structural protein is E.

8. The method of claim 1, wherein the cis-acting RNA sequence is PS9 or T20.

9. The method of claim 1, wherein the cis-acting RNA sequence is PS9.

10. The method of claim 1, wherein the initial and secondary cells are from human, bat, bird, or dog.

11. The method of claim 1, wherein the initial and secondary cells are from a cell line.

12. The method of claim 1, wherein the initial and secondary cells are kidney cells.

13. The method of claim 1, wherein the secondary cells overexpress the ACE2 and/or TMPRSS2.

14. The method of claim 1, wherein the initial and secondary cells are human.

15. The method of claim 2, wherein the selection pressure is a therapeutic compound.

16. The method of claim 15, wherein the therapeutic compound is an antibody or sera from a human following infection or vaccination.

17. The method of claim 15, wherein the therapeutic compound is a small molecule, a protein, a peptide, a polynucleotide, a polysaccharide, an oil, a solution or a plant extract.

18. The method of claim 17, wherein the small molecule is an antiviral compound.

19. The method of claim 1, wherein prior to sequencing the viral sequences in d), the RNA is extracted, RT-PCT is performed on said RNA, and sequencing adapters are added to the DNA obtained from the RT-PCR.

20. The method of claim 3, wherein the neutralizing antibodies are from sera from a human following infection or vaccination.
